(19) United States
(12) Patent Application Publication
(10) Pub. No.: US 2025/0025191 A1
(43) Pub. Date: Jan. 23, 2025

(54) EMBOLUS REMOVAL APPARATUS AND METHODS

(71) Applicant: VASCULAR CLARITY LTD., Tel Aviv (IL)

(72) Inventors: Abraham PENNER, Tel Aviv (IL); Alon BEN YOSEF, Ramot Menashe (IL)

(21) Appl. No.: 18/708,082

(22) PCT Filed: Nov. 30, 2022
(86) PCT No.: PCT/IL2022/051275

§ 371 Date: May 7, 2024

Related U.S. Application Data
(60) Provisional application No. 63/285,501, filed on Dec. 3, 2021.

(51) Int. Cl. A61B 17/221; A61B 17/22

(52) U.S. Cl. CPC A61B 17/221; A61B 2017/22079; A61B 2017/2215

(57) ABSTRACT

The present disclosure relates to devices and methods for removing emboli from one or more blood vessels. In an example, an embolus removal apparatus includes a delivery catheter, an embolus collection basket connected to a basket shaft extending through the delivery catheter, a retrieval shaft extending through the delivery catheter, and at least one capture mechanism configured to couple an embolus to a distal portion of the retrieval shaft. The retrieval shaft is can be navigated, with the embolus carried thereby, toward and into a cavity of the basket, and can optionally release the embolus in the basket and optionally be navigated to capture and direct another embolus, if required, to the basket. Once all emboli are collected in the basket's cavity, the apparatus can be retrieved from the patient's body with the emboli captured therein.

### FIELD

The present disclosure relates to devices and methods for removing unwanted material from the body of a subject, and more specifically to removal of emboli from the vasculature of a mammal subject.

### BACKGROUND

Pulmonary embolism (PE) is an embolus in the lung arteries which most often migrates from the legs and is associated with deep vein thrombosis (DVT). PE can also be a complication of other viral or inflammatory diseases. For example, COVID-19 may lead to venous and arterial thromboembolism disease due to excessive inflammation, hypoxia, immobilization and diffuse intravascular coagulation.

PE is not always easy to diagnose as its symptoms are similar to other diseases (dyspnea, tachypnea, pleuritic pain, calf or thigh pain, calf or thigh swelling, tachycardia and cough). PE can lead to high morbidity and mortality rates. In the US and Europe, PE is considered the third cause of cardiovascular death (after acute MI and stroke). The severity of PE and the patient's presentation drive treatment selection and the choice of care plan. Treatment options for PE can include anticoagulants to prevent additional embolus grow or new embolus formation, and means for removing or dissolving the embolus, such as systemic thrombolytics therapy, catheter-based therapy and/or surgery.

Thrombolytic treatment for PE patient is based on the administration of tissue plasminogen activator (tPA). This drug converts plasminogen to plasmin in the coagulation cascade, which results in fibrinolysis. While being an effective therapy to PE, acute ischemic stroke and myocardial infraction, systemic thrombolytic therapy it is also associated with around 10% of bleeding events including about 3% of hemorrhagic stroke. Due to the potential catastrophic complications associated with hemorrhagic stroke, the relevant guidelines for PE treatment recommend not to use thrombolytic therapy unless the patient is at a life-threatening risk due to PE. This recommendation limits the use of thrombolytic therapy to only about 5% of PE patients.

To reduce this systemic effect, numerous catheter-directed therapies were developed, including local delivery of thrombolytic agents which may include ultrasound, use of pressurized saline injection, or catheter-led mechanical disruption of the embolus. While local delivery of tPA using catheter was shown to be more effective and safer than systemic thrombolysis, catheter can locally deliver tPA only to the main arteries, and requires an invasive procedure of catheterization with the risk associated with it. Additional potential drawback include significant procedural costs, and requirement for dedicated expertise and equipment (e.g. interventional cardiologist and catheterization suite). As a result, the use of catheter-based thrombolysis is limited to the highest risk group of PE patients.

Surgical intervention is considered, due to the high risk associated with the procedure, only for hemodynamically instable patients.

Mechanical debulking and/or aspiration devices can be used to remove an embolus or other obstruction. These mechanical techniques can either macerate, aspirate, or a combination thereof in order to remove the blood embolus. An advantage of mechanical therapy is that it can remove thrombus directly from the blockage area and immediately eliminates the obstruction. Thus, this approach may be superior to thrombolytic agents in some cases.

Several catheter-based technologies were developed for debulking an embolic obstruction from large blood vessels such as the vena cava and the pulmonary arteries, including vacuum aspiration, mechanical maceration or fragmentation, possibly in combination with aspiration, or mechanical retrieval device. Another technology suggested is rheolytic thrombectomy, using high velocity saline jets to create a Venturi effect and draw the fragments of the embolus into the catheter. This method poses risk of perforating body lumen and hemolysis. In some cases, such debulking methods may be used in combination with thrombolysis.

Despite apparent superiority over alternative solutions, current mechanical therapies have some major limitations. Primarily, most of these technologies require a large catheter having a similar size to that of the removable embolus.

For example, an embolus in the left or right pulmonary artery (PA), may have a diameter of 12-20 mm. Typically, such a thrombus can contain about 50% of liquid (or less for a well-organized clot), which may be squeezed out of the embolus, reducing its diameter to about 6-10 mm. Still, for removing such an embolus, an 18 to 30 Fr catheter is required to be positioned in proximity to the embolus. However, even aspiration via a large bore catheter may break smaller fragment of the embolus, that may flow downstream, potentially causing distal occlusion which may lead to hemodynamic instability. Navigation of such a large profile catheter is difficult and challenging, especially if the path along the vasculature is as tortuous as the path leading to the PA. Reaching intravenously to the PA requires passing through the vena cava, the tricuspid valve, the right atrium, the right ventricle, crossing the pulmonic valve to the main pulmonary artery, and then navigating to the target artery.

Navigation of such a large catheter profile imposes a significant risk to the heart structures, such as to the chambers, valves, and blood vessels crossed by the catheter. The challenge to navigate a large catheter to the pulmonary tree results in limited utilization of embolus mechanical retrievers to treat PE. Similar limitation exists in other body lumens such as occluded cerebral arteries leading to ischemic stroke. Moreover, a large-profile catheter may also partially or fully occlude blood flow during the procedure, thus limiting procedural duration before patients may become hemodynamically instable.

Current small size devices are unsuitable for removal of large blood embolus as a whole, and often require breaking of the embolus into smaller pieces prior to removal thereof. Embolus fragments can flow downstream, potentially causing distal occlusion which may lead to hemodynamic instability. Overall, using current small catheters is risky, ineffective and time consuming.

In some situations, the embolus removed is highly organized and the amount of embolus is substantial, making it difficult to remove the embolus through a small catheter lumen.

Catheter-based removal of blood embolus, from the pulmonary arteries, is also limited by the small distal working space due to frequent branching of the pulmonary tree. Most current devices require a long straight lumen segment to properly function, which significantly limits their effectiveness in branching vasculature structures such as the pulmonary arteries or the cerebral circulation.

It is evident that current treatment options, available to patients with blood embolus in branching vasculature structures, have limited effectiveness. While most of these patients receive anticoagulation therapy, such therapy mainly limits embolus growth, but does not actively dissolve it. Thrombolytic therapy takes several hours to start dissolving the embolus, has limited effectiveness for treating large or organized embolus, and poses a significant risk of major bleeding. Effective catheter embolectomy requires a large profile catheter, which is hard to navigate to the pulmonary arteries and poses a risk of harming the heart structures and the blood vessels. Low profile catheter thrombectomy cannot effectively remove material from large vessels, it is time consuming and may cause distal embolization. Additionally, these devices are hard to operate in a complex vascular structure. While surgical embolectomy can be effective, it is highly invasive and is associated with high rates of morbidity and mortality.

### SUMMARY

Described herein are examples of devices and methods that utilize low profile catheter treatment for safer, and more effective removal of large blood embolus, so as to restore immediate blood flow.

According to some aspects of the disclosure, there is provided an embolus removal apparatus comprising a delivery catheter defining a delivery catheter lumen, and an embolus collection basket. The embolus collection basket is coupled to a basket shaft which extends through the delivery catheter lumen, and comprises porous walls that surround a collection basket cavity, extending between an embolus deposit opening and a permanently closed end. The embolus removal apparatus further comprises a retrieval shaft extending through the delivery catheter lumen and comprising a distal portion. The retrieval shaft further comprises at least one capture mechanism, configured to couple an embolus to the distal portion of the retrieval shaft. The retrieval shaft and the basket shaft are each axially movable relative to the delivery catheter. The collection basket is configured to transition between a collapsed state and an expanded state. The retrieval shaft is movable between a first position of its distal portion distanced from the collection basket cavity, to a second position of its distal portion inside the collection basket cavity. The at least one capture mechanism is configured to maintain engagement between an embolus and the distal portion of the retrieval shaft, during translation of the distal portion from the first position to the second position.

According to some aspects of the disclosure, there is provided a method for removing at least one embolus from at least one blood vessel. The method comprises advancing a delivery catheter of an embolus removal apparatus towards a site of treatment which includes at least one embolus. The method further comprises extending an embolus collection basket past a distal end of the delivery catheter, and expanding the embolus collection basket distally to the delivery catheter. The method further comprises extending a retrieval shaft past the distal end of the delivery catheter and towards the at least one embolus, and capturing the at least one embolus by a capture mechanism. The method further comprises guiding the retrieval shaft with the at least one embolus towards and into a collection basket cavity of the collection basket through an embolus deposit opening. The method further comprises retrieving the embolus removal apparatus.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying Figures.

### BRIEF DESCRIPTION OF THE FIGS.

Some examples of the invention are described herein with reference to the accompanying Figures. The description, together with the Figures, makes apparent to a person having ordinary skill in the art how some examples may be practiced. The Figs. are for the purpose of illustrative description and no attempt is made to show structural details of an example in more detail than is necessary for a fundamental understanding of the invention. For the sake of clarity, some objects depicted in the Figures are not to scale.

In the Figures:

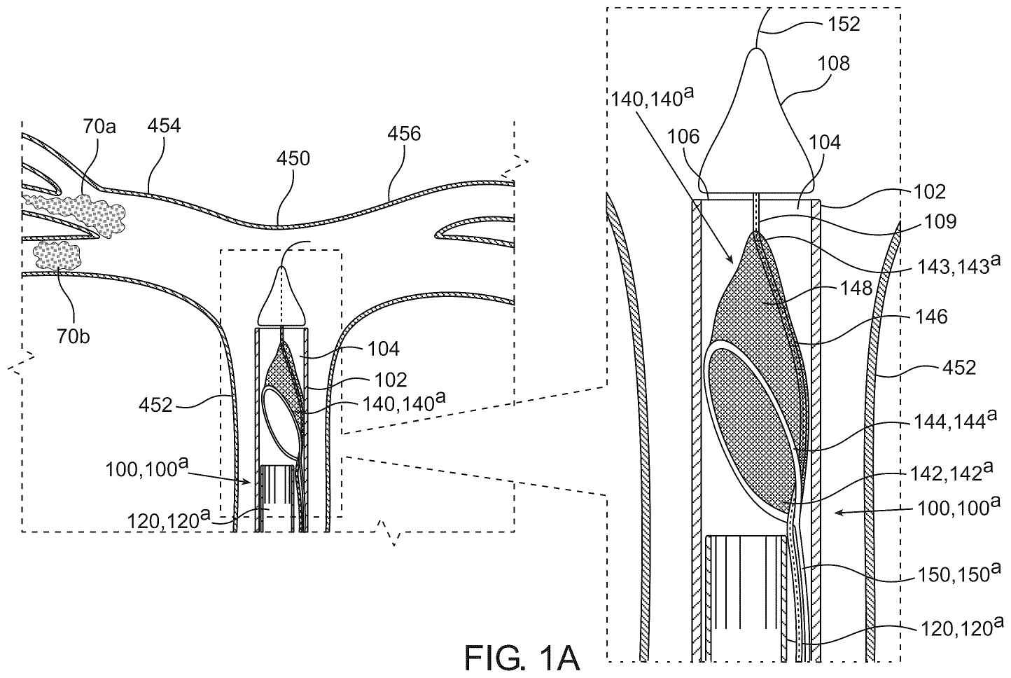

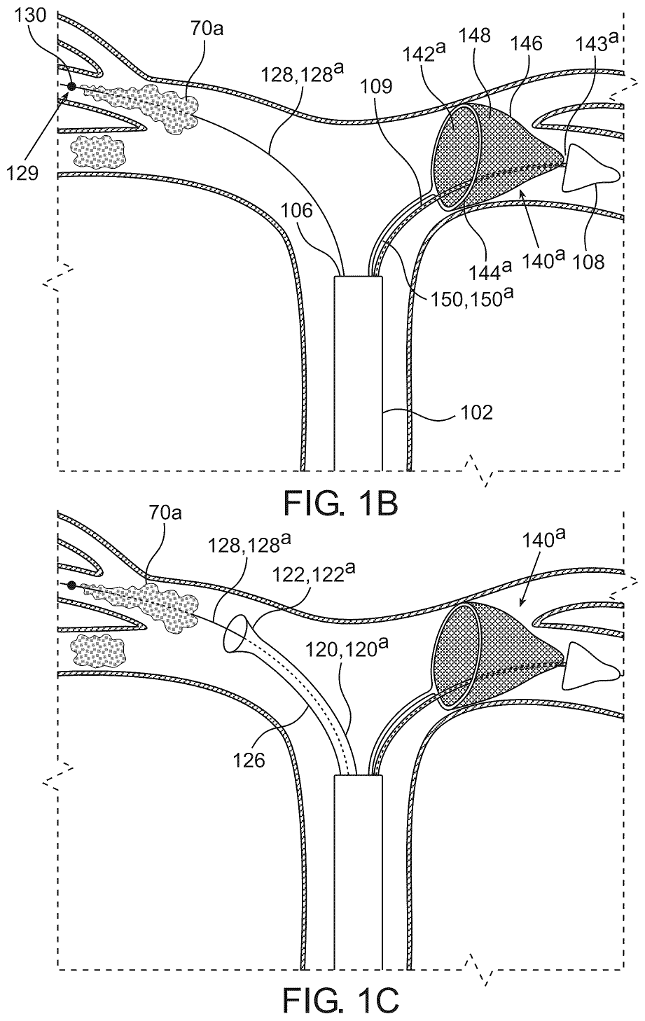

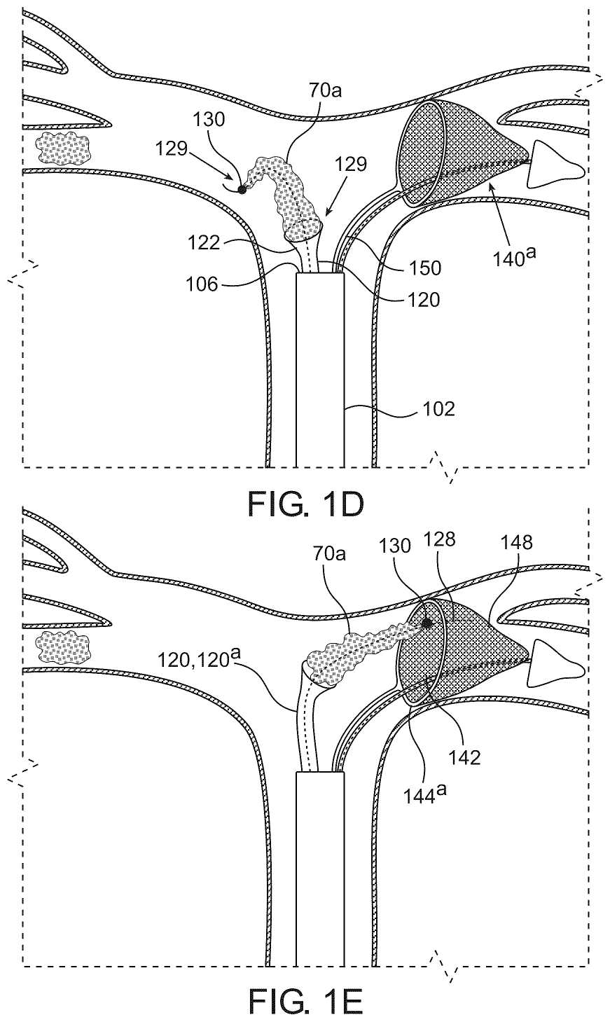

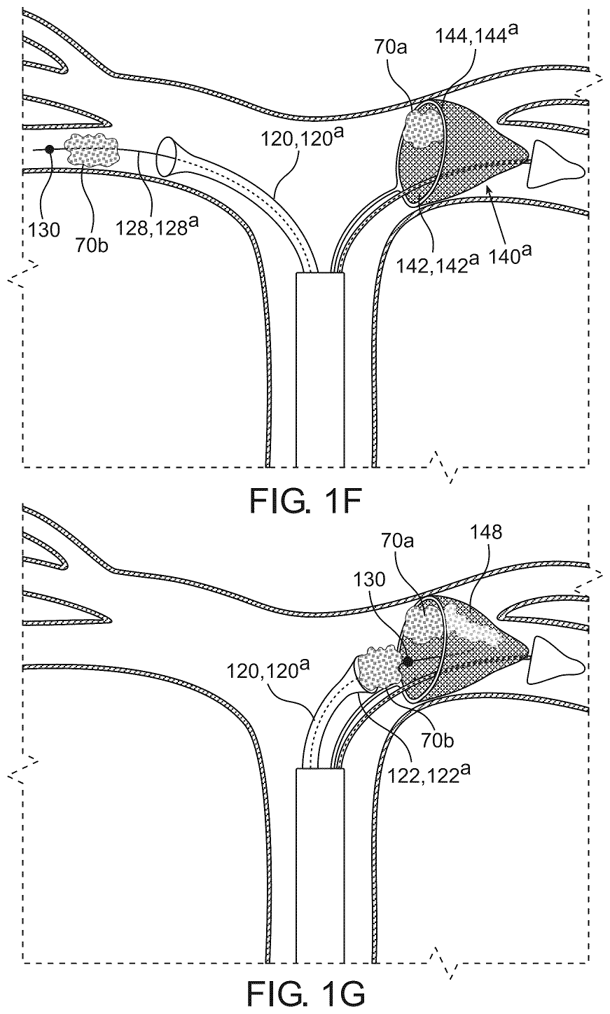

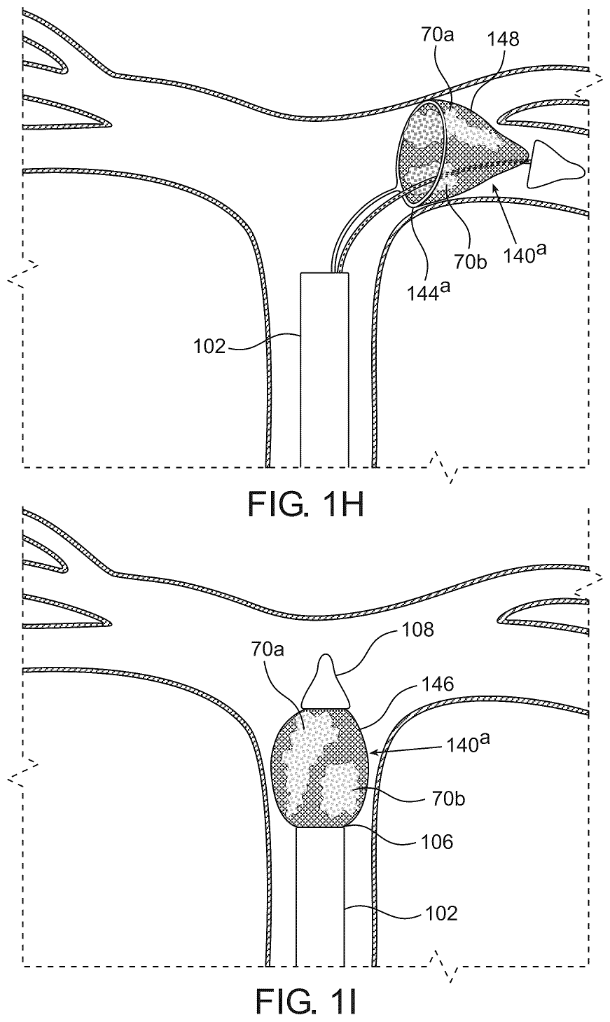

FIGS. 1A-1I are schematic illustrations of an exemplary embolus removal apparatus, shown in sequential stages of an exemplary embolus removal procedure.

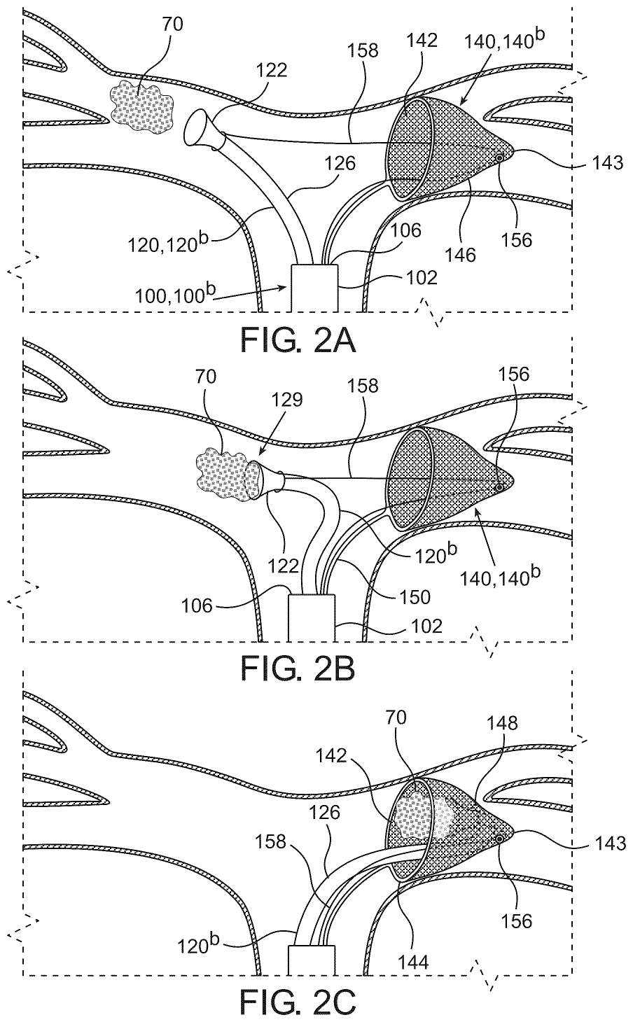

FIGS. 2A-2C are schematic illustrations of exemplary embolus removal apparatus equipped with a tether coupled to a retrieval shaft, in sequential stages of capturing and delivering an embolus into a collection basket.

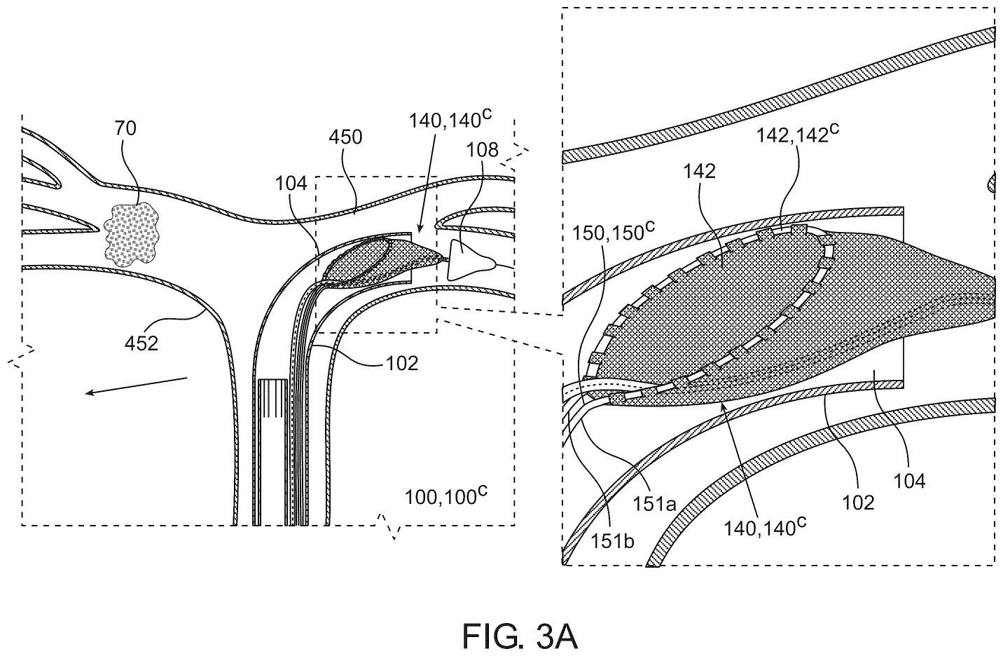

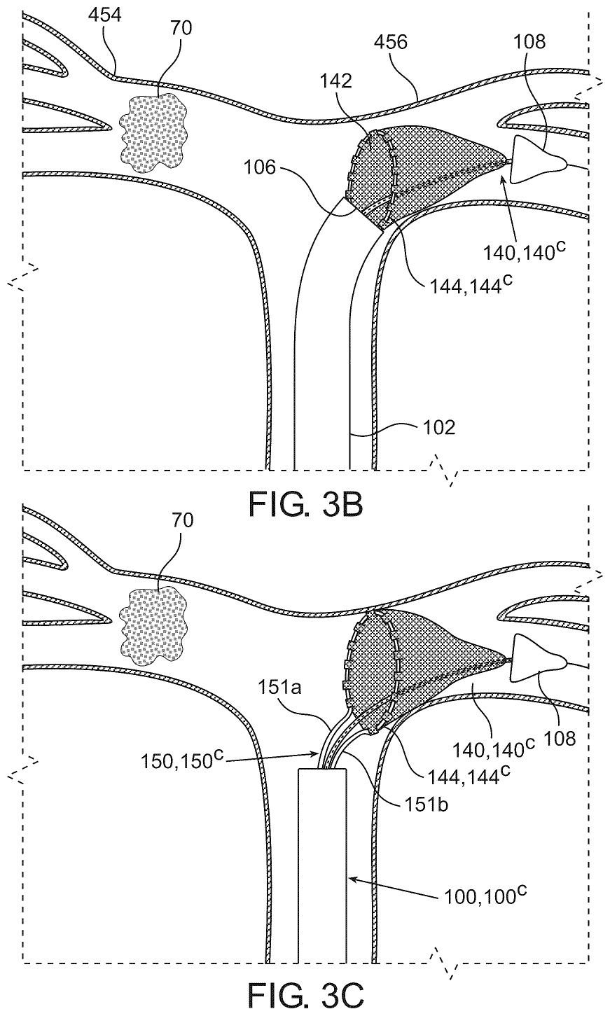

FIGS. 3A-3C are schematic illustrations of an exemplary embolus removal apparatus, in sequential stages of expanding a collection basket within a junction of the pulmonary artery.

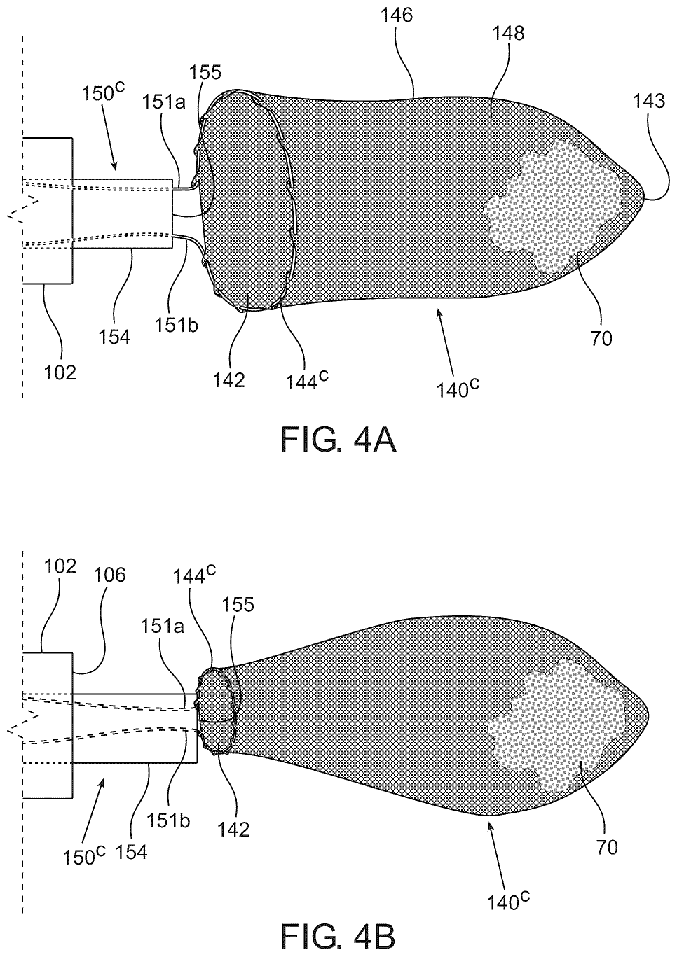

FIGS. 4A-4B illustrate a collection basked that includes wire portions utilized to transition an embolus deposit opening thereof, from an open state (FIG. 4A) to a closed state (FIG. 4B).

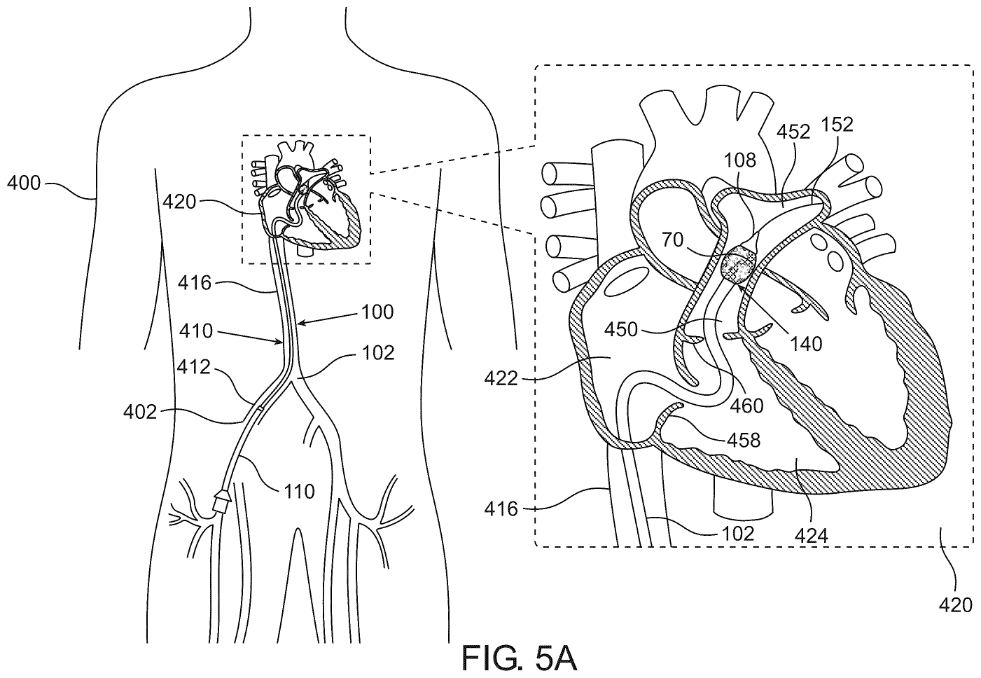

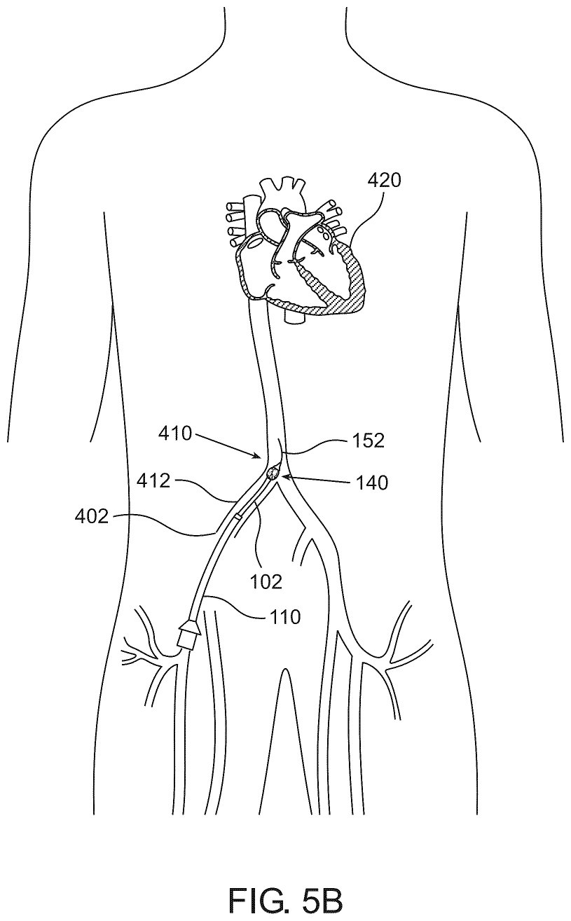

FIGS. 5A-5B show exemplary steps in retraction of an embolus removal apparatus from a patient's body.

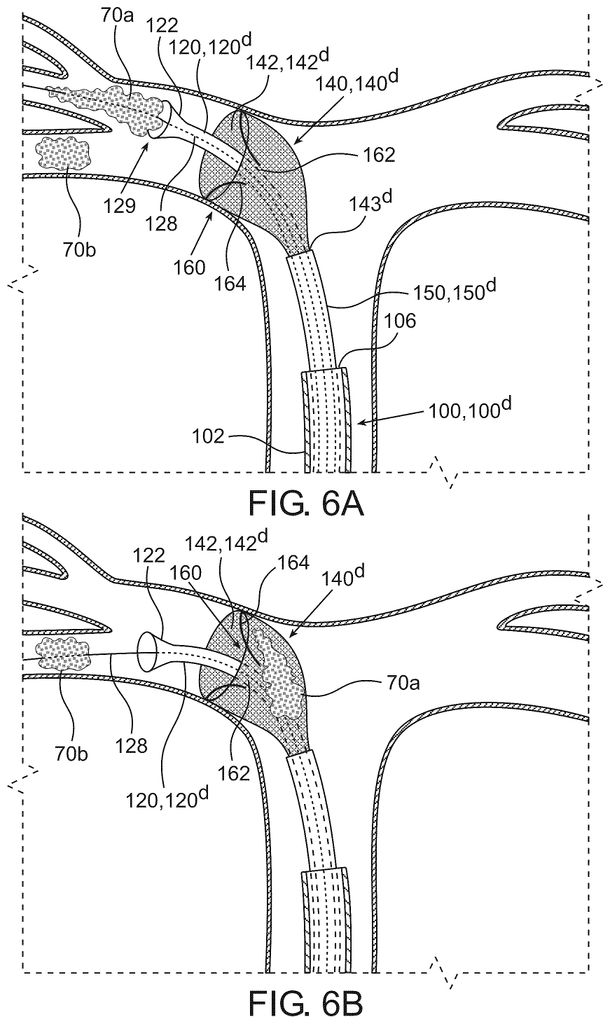

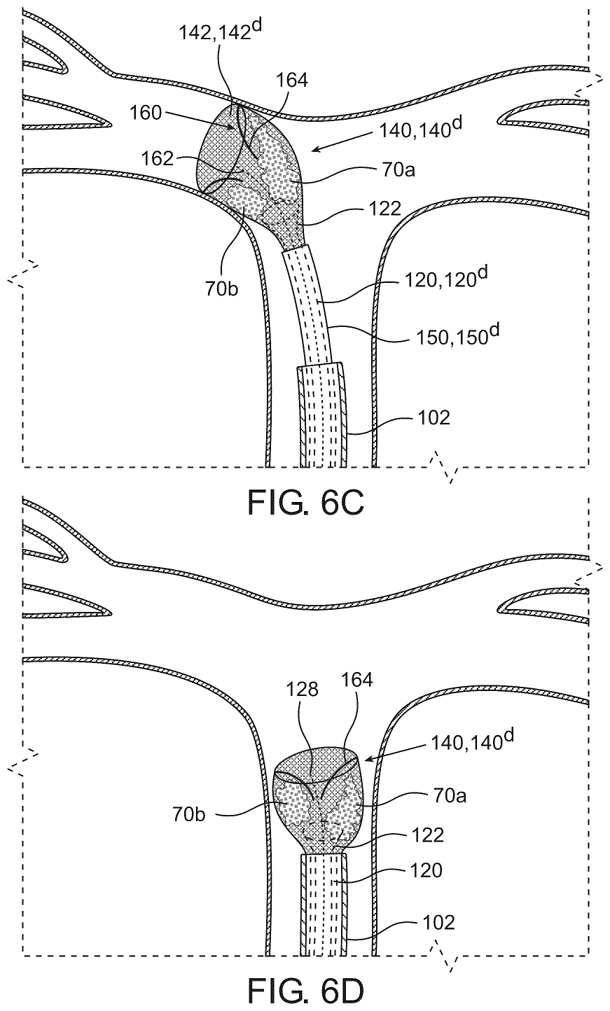

FIGS. 6A-6D are schematic illustrations of an exemplary embolus removal apparatus equipped with a retrieval catheter extendable through an embolus collection basket, in sequential stages of capturing and delivering an embolus into a collection basket.

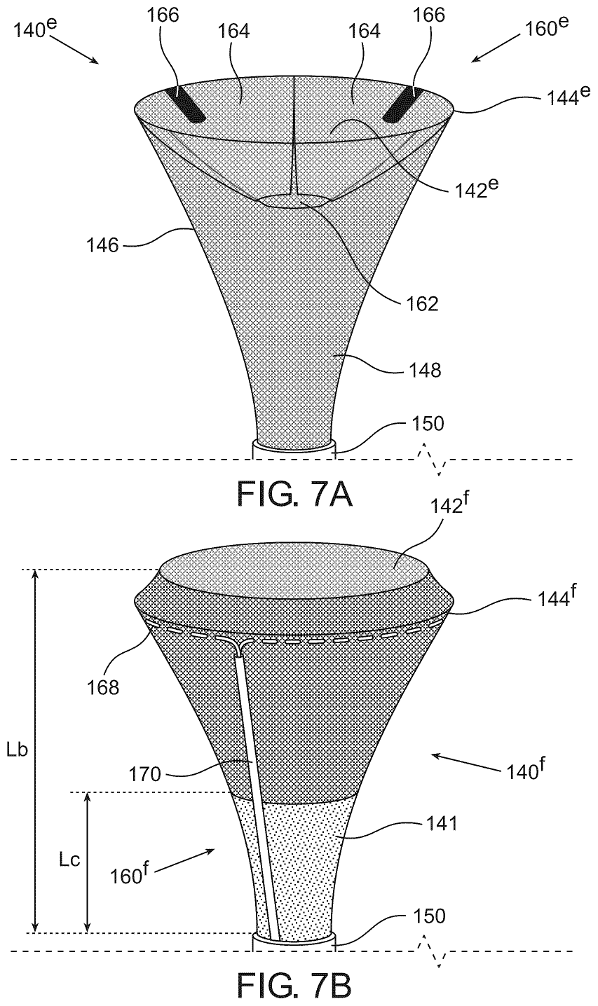

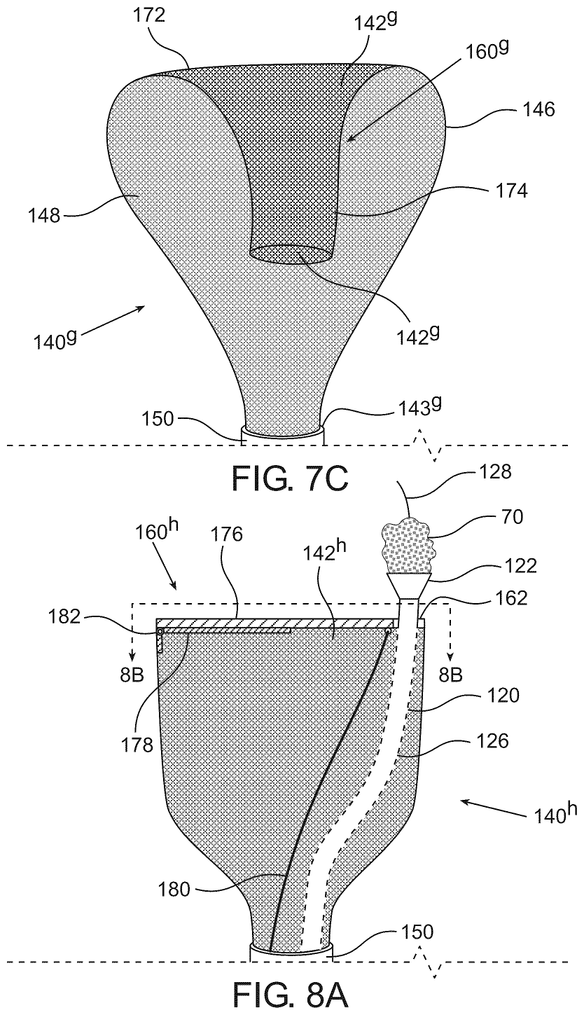

FIG. 7A shows an exemplary basket comprising a closing mechanism that includes cover flaps.

FIG. 7B shows an exemplary collection basket comprising a closing mechanism that includes a drawstring.

FIG. 7C shows an exemplary collection basket that includes an inverted neck.

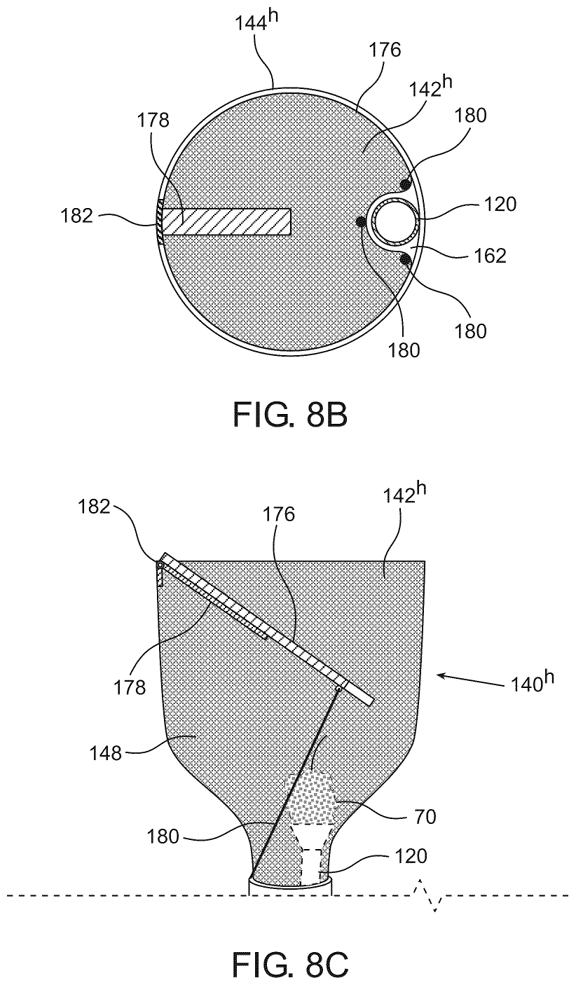

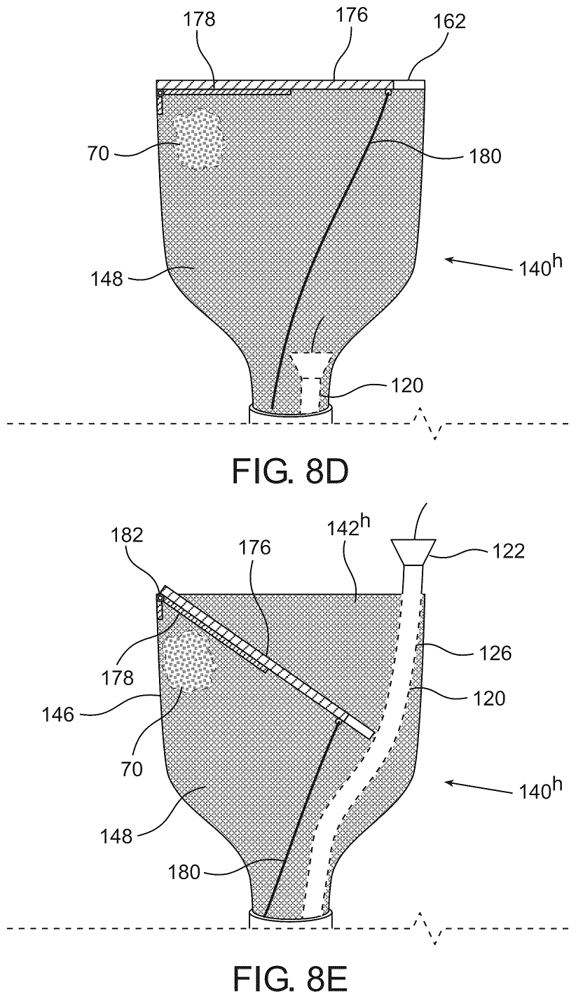

FIGS. 8A-8E illustrate an exemplary basket comprising a closing mechanism that includes a hinged lid, shown in various stages of utilization thereof.

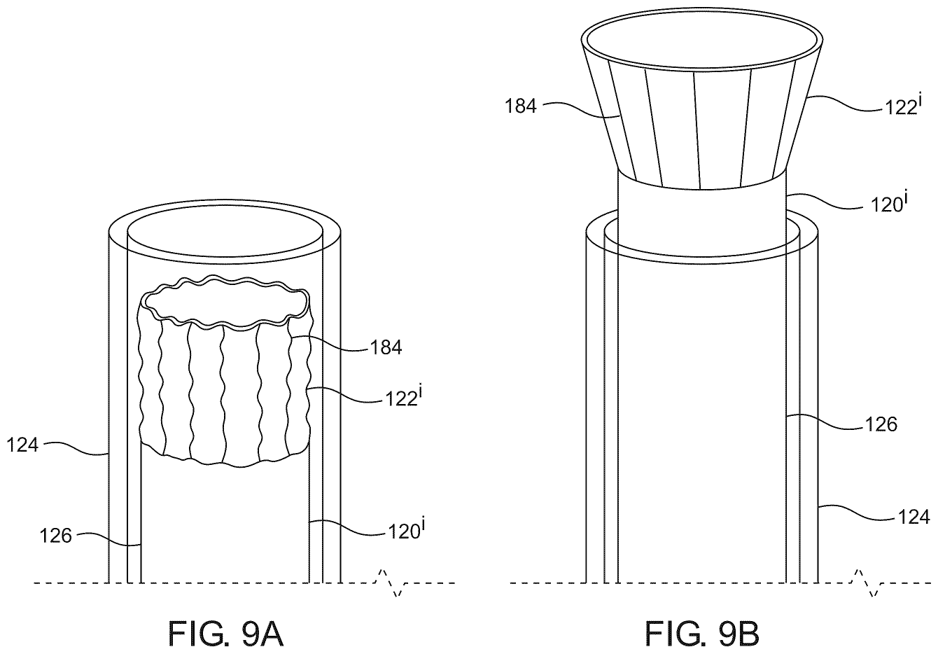

FIGS. 9A-9B illustrate an expandable distal portion of a retrieval shaft, shown in a collapsed configuration (FIG. 9A) and an expanded configuration (FIG. 9B).

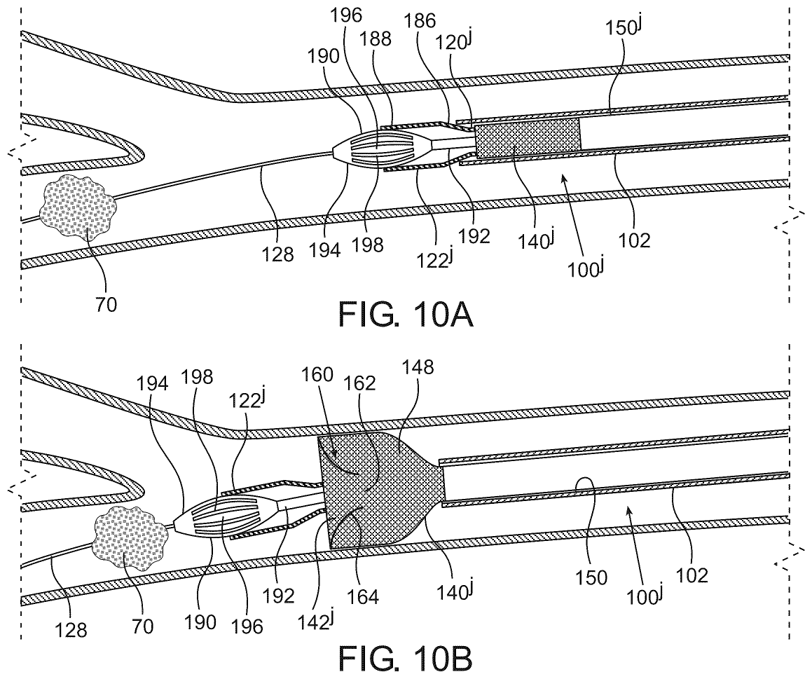

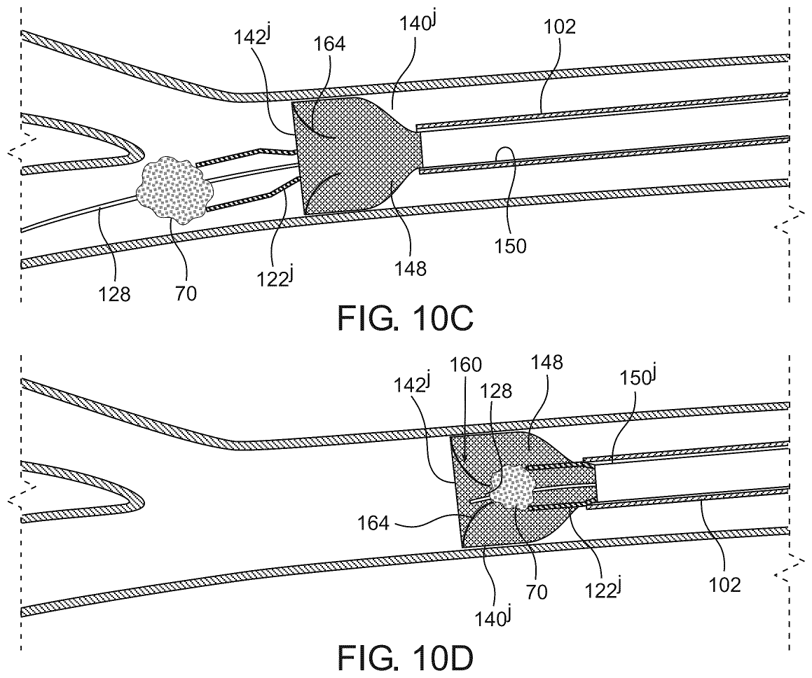

FIGS. 10A-10D are schematic illustrations of an exemplary embolus removal apparatus equipped with an expendable nosecone, in sequential stages of capturing and delivering an embolus into a collection basket.

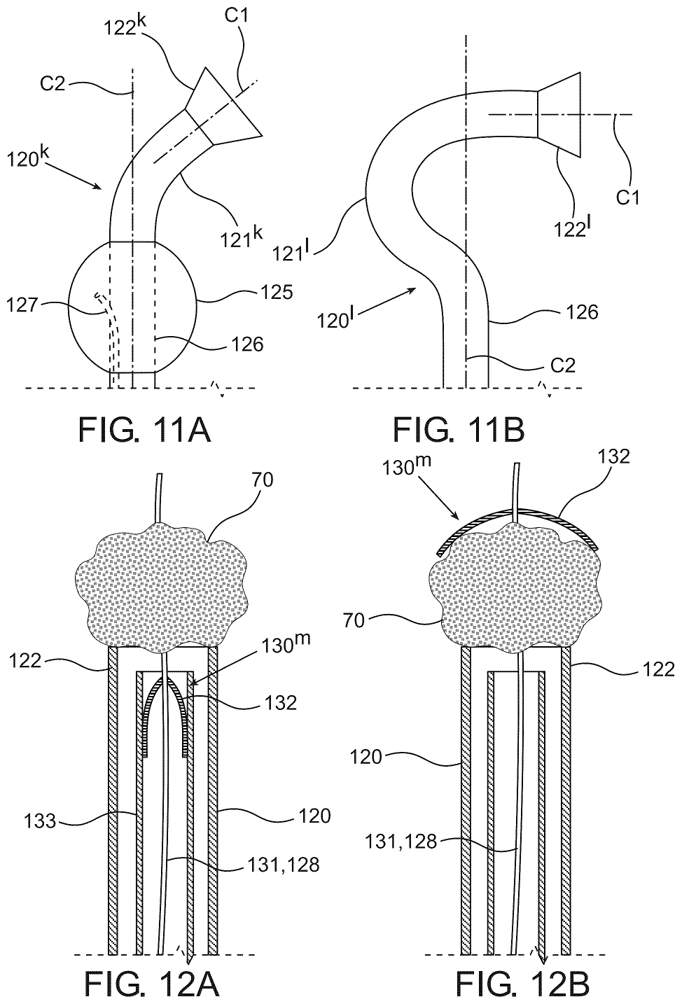

FIGS. 11A-11B show exemplary retrieval shafts.

FIGS. 12A-12B illustrate an exemplary embolus-capture element that includes an expandable plate, shown in a collapsed configuration (FIG. 12A) and an expanded configuration (FIG. 12B).

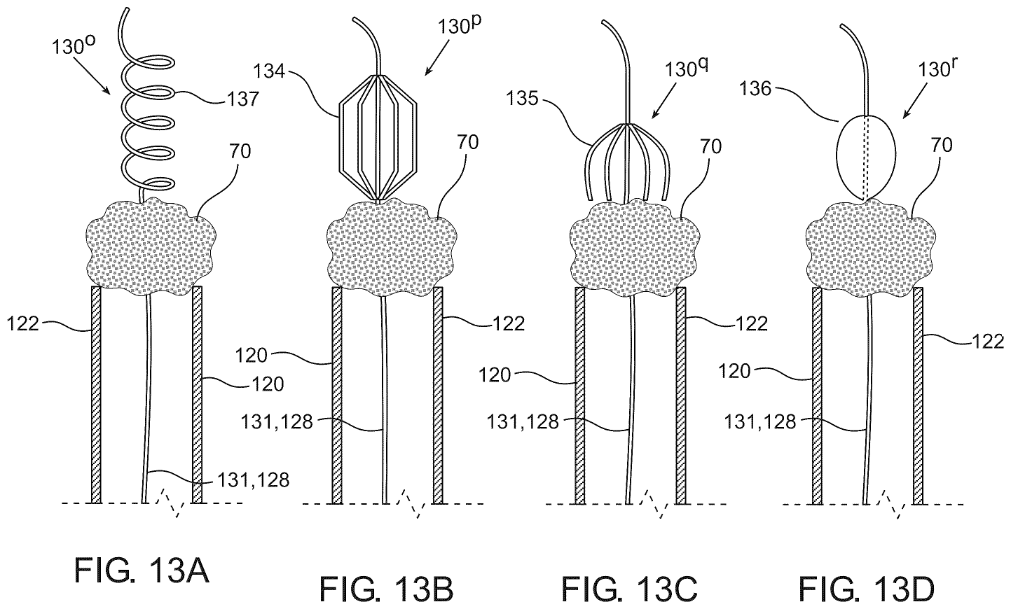

FIG. 13A shows an exemplary embolus-capture element that includes a helical coil.

FIG. 13B shows an exemplary embolus-capture element 130P that includes an expandable frame.

FIG. 13C shows an exemplary embolus-capture element 130^{q} that includes a proximally forked arrangement.

FIG. 13D shows an exemplary embolus-capture element 130^{r} that includes an inflatable pulling balloon.

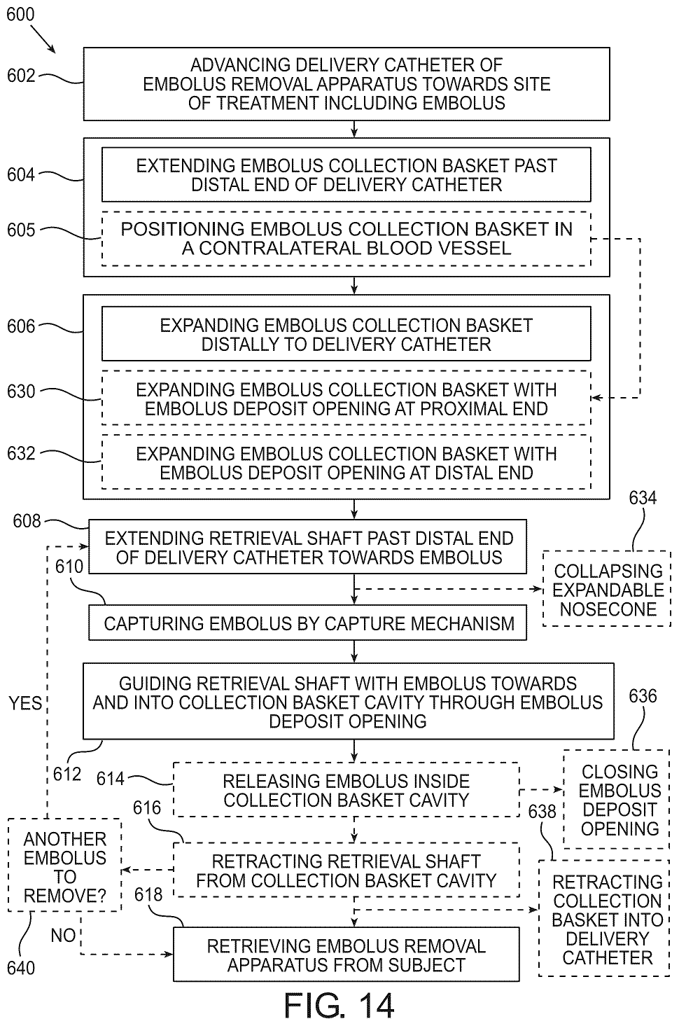

FIG. 14 is a flowchart depicting examples a method of removing at least one embolus from at least one blood vessel.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached Figs. may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms “distal” and “proximal” as used herein, unless expressly stated otherwise, refer to a position which is further or nearer, respectively, along the advancement route of the embolus removal apparatus into the body of the subject.

The terms “downstream” and “upstream” as used herein, unless expressly stated otherwise, refer to a position which is further or nearer, respectively, along a blood vessel in the blood flow direction.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

As used herein, the unit “Fr” represents French scale unit for measuring the outer diameter of a catheter, shaft, or tube. As is well known in the art, the relation between the Fr unit and the millimeter unit is 3 Fr=1 mm.

The terms embolus and clot, as used herein, are interchangeable, and refer to any type of blocking material that obstructs a blood vessel.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript.

In order to avoid undue clutter from having too many reference numbers and lead lines on a particular drawing, some components will be introduced via one or more drawings and not explicitly identified in every subsequent drawing that contains that component.

FIGS. 1A-1I are schematic illustrations of an exemplary embolus removal apparatus 100, located within a pulmonary artery (PA) of a patient 400 (patient 400 indicated in FIG. 5A, for example), in sequential stages of an exemplary embolus removal procedure. For the sake of convenience, the components of the embolus removal apparatus 100 will be described in relation to FIGS. 1A to 1I, as a whole. Embolus removal apparatus 100 includes a delivery catheter 102 proximally extending from a distal end 106. Delivery catheter 102 defines a delivery catheter lumen 104 through which devices, instruments and other material therethrough can be delivered. Extending through delivery catheter lumen 104 is a basket shaft 150 attached to an embolus collection basket 140 at its distal end. The embolus removal apparatus 100 further comprises a retrieval shaft 120 that can also extend, in some examples, through delivery catheter lumen 104.

In some examples, embolus removal apparatus 100 can further include a nosecone 108 at the distal end of delivery catheter 102, configured to facilitate advancement of delivery catheter 102 through the patient's vasculature. A nosecone shaft 109 can extend proximally from the nosecone 108, defining a guidewire lumen configured to allow passage of a delivery guidewire 152 therethrough, over which the embolus removal apparatus 100 can be advanced to a desired site of treatment, such as within the PA.

Collection basket 140 includes permeable/porous basket walls 146, which are shaped to form a collection basket cavity 148, enclosed by walls 146, and an embolus deposit opening 142 configured to transition between an open state and a closed state, which is sized, in in its open state, to allow passage of an embolus 70 therethrough, into collection basket cavity 148, while in the closed state, it may be substantially closed or narrowed to a size configured to prevent emboli trapped within the cavity 148 from escaping therethrough. Rim 144 surrounds embolus deposit opening 142. The porous basket walls 146 extend from the rim 144 around embolus deposit opening 142, to a permanently closed end 143 of the basket 140, defined as the end opposite to the embolus deposit opening 142, and can be an integral end portion of porous basket walls 146, and serve as a porous end which prevents embolus 70 trapped in cavity 148 from distally advancing beyond permanently closed end 143.

In some examples, when the embolus deposit opening 142 is in the open state, the porous basket walls 146 can taper from a greater diameter defined by embolus deposit opening 142 to a narrower tip formed by the permanently closed end 143. A collection basket 140 according to any example disclosed herein, can be at least partially conically shaped in some examples, in an expanded or deployed state of basket 140. In some examples, embolus deposit opening 142 does not necessarily define the largest diameter of the collection basket 140, and the collection basket 140 may assume a spherical shape, a cylindrical shape, a zeppelin shape, an hourglass shape, or any other shape in an expanded or deployed state thereof. According to some examples, embolus deposit opening 142 may be relatively narrow (i.e., define a relatively small diameter), and be made of a stretchable material such that a collected embolus 70 can be passed tightly therethrough.

Various exemplary implementations for embolus removal apparatus 100 and components thereof can be referred to, throughout the specification, with superscripts, for case of explanation of features that refer to such exemplary implementations. It is to be understood, however, that any reference to structural or functional features of any assembly, apparatus or component, without a superscript, refers to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, embolus removal apparatus 100^{a}, illustrated in FIG. 1A-1I, is an exemplary implementation of embolus removal apparatus 100, and thus includes all of the features described for embolus removal apparatus 100 throughout the current disclosure, except that the embolus deposit opening 142^{a} is at the proximal end of collection basket 140^{a} and the permanently closed end 143^{a} is at the distal end of collection basket 140^{a}.

During delivery to the site of treatment, the collection basket 140^{a} can be retained in a compressed, collapsed or folded configuration within delivery catheter lumen 104, such that the permanently closed end 143^{a} is distally oriented, toward the distal end 106 of delivery catheter 102. Rim 144^{a} is connected to basket shaft 150^{a} which extends through delivery catheter lumen 104 and is operational for advancing and retracting collection basket 140. As further shown, retrieval shaft 120^{a} of embolus removal apparatus 100^{a} extends through delivery catheter lumen 104, in parallel to basket shaft 150^{a} and collection basket 140^{a}, without extending through collection basket 140^{a} or its embolus deposit opening 142^{a} during delivery of the apparatus 100^{a} to the site of treatment. This separation allows each of the basket 140^{a} via basket shaft 150^{a}, and retrieval shaft 120^{a}, to be advanced and navigated past the distal end 106 of delivery catheter 102 at non-equal and non-similar directions if desired, including each being guided or deployed at an opposite direction relative to the other.

In some examples, nosecone 108 can be coupled to the distal end of collection basket 140, such as to the permanently closed end 143^{a} of collection basket 140^{a}.

In some examples, nosecone 108 is distal to collection basket 140, and can be optionally movable (e.g., axially movable) with respect to basket 140, such as with respect to the permanently closed end 143^{a} of collection basket 140^{a}. In some examples, the nosecone shaft 109 can extend through the basket opening 142 and basket cavity 148, such as towards and through the permanently closed end 143^{a} at the distal end of basket 140^{a}. In such examples, the nosecone shaft 109 can extend through a distal opening that can be formed at the permanently closed end 143^{a}, sized to allow extension of the nosecone shaft 109 therethrough. When the nosecone shaft 109 extends through the basket cavity, it may be desirable to retain it in close proximity to a sidewall of collection basket 140, such as an inner surface of porous basket walls 146, so as to prevent it from interfering with passage of other material and components, such as emboli and/or shafts or catheters for carrying emboli into the basket 140.

In some examples, the basket shaft 150, such as basket shaft 150^{a}, is pre-shaped so as to be curved at least along a portion thereof, such as a distal portion extending from the rim 144. A curved basket shaft 150 allows it to better conform to the anatomical curvature when the basket is pushed out of delivery catheter lumen 104, such as along the path from the main pulmonary artery 450 to a branched target vessel, such as the LPA 456. The basket shaft 150 can be made of a shape-memory material, such as Nitinol or a shape-memory polymer, pre-shaped to assume a curved shape in a free state thereof. Basket shaft 150 can be flexible enough to assume a relatively straight configuration when positioned inside the delivery catheter 102 during advancement through the patient's vasculature, and once deployed or advanced distally from delivery catheter lumen 104, for example, the basket shaft 150 can be free to assume its pre-shaped curved configuration. In some examples, a cover-sheath, similar for example to collection basket cover-sheath 154 shown in FIG. 4A, can be disposed over a pre-formed wire or other member of the basket shaft 150 during delivery of the embolus removal apparatus 100, forcing it to assume a relatively straight configuration, and once deployed or advanced from the collection basket cover-sheath 154, the curved wire or other shaft member can be free to assume its pre-shaped curved configuration.

FIG. 1A shows a sectional view of exemplary embolus removal apparatus 100^{a} advanced into the pulmonary trunk 452 of main pulmonary artery (MPA) 450. Retrieval shaft 120 and collection basket 140 are located within delivery catheter lumen 104, in a retracted, and optionally also a collapsed, state. Nosecone 108 is located in the illustrated example at the distal end 106 of delivery catheter 102. FIG. 1A illustrates an exemplary pathology in which a first embolus 70a and a second embolus 70b are situated in two branches of the right pulmonary artery (RPA).

In some examples, delivery catheter 102 can have a diameter of about 5 Fr to about 18 Fr. In some examples, delivery catheter 102 can have a diameter of about 7 Fr to about 15 Fr. In some examples, delivery catheter 102 can have a diameter of about 8 Fr to about 12 Fr. This diameter can be utilized, in some implementations, when removing an embolus 70 from a large blood vessel, such as the left pulmonary artery (LPA) or the RPA, which may typically have a diameter of about 13 to 22 mm. In some implementations, the embolus removal apparatus 100 can be used for removing at least one embolus 70 from a small vessel. As will be described in greater details throughout FIGS. 1A-1I, delivery catheter 102 will not usually extend all the way to embolus 70, but rather terminate proximally thereto, from which retrieval shaft 120 is extended toward embolus 70. In some examples, retrieval shaft 120 is smaller in diameter by between about 2 Fr and 8 Fr than delivery catheter 102. For example, when a 12 Fr delivery catheter 102 is used, the size of the retrieval shaft 120 can be in the range of about 6 Fr to about 9 Fr.

According to some examples, any of the delivery catheter 102, the retrieval shaft 120, and/or the nosecone shaft 109, can be created from extruded tubes made of nylon, nylon derivates (e.g., 6/6, 6/12, or any other nylon derivate), Pebax®, Polyurethane, PTFE, TFE, PEEK or any other polymer used for catheter extrusion known in the art. According to some examples, the tube of delivery catheter 102 and/or retrieval shaft 120 may include a metallic coil, braid, hypotube, or a combination thereof, wherein the metal may include stainless steel, nickel-titanium, or any other relevant metal known in the art for reinforcing catheters. Delivery catheter 102, retrieval shaft 120, or any other shaft or component of embolus removal apparatus 100, may include one or more radio-opaque markers or additives for allowing tracking of the advancement and/or current location of the component within the patient's vasculature. Radio-opaque additives can include, for example, Barium sulfate or Bismuth based molecules, e.g. Bismuth trioxide.

To reduce injury or unwanted friction with the blood vessel or other body structures, delivery catheter 102 and/or retrieval shaft 120 can include a hydrophobic coating, a hydrophilic coating, or a lubricant. Nosecone 108 can have a tapering shape extending from a narrower distal end to a wider proximal diameter of the nosecone, and can be coated or made of a material allowing smooth and atraumatic advancement through the patient's vasculature. Some exemplary configurations and materials for the nosecone are described herein in relation to nosecone 190 which is described below with reference to FIGS. 10A to 10D. These configurations and materials may be equally applied with regard to nosecone 108.

In some examples, delivery catheter 102 can be implemented as a multi-lumen shaft defining more than one lumen through which components of the embolus removal apparatus 100 can extend, optionally each through a separate lumen. For examples, a multi-lumen delivery catheter 102 of an embolus removal apparatus 100^{a} can define one lumen through which the basket shaft 150 can extend, and a separate, optionally parallel, lumen, through which the retrieval shaft 120 can extend. In some examples, a delivery guidewire 152 can pass alongside any of the basket shaft 150 and/or retrieval shaft 120. For example, a nosecone shaft 109 defining a guidewire lumen through which guidewire 152 can extend, may pass alongside basket shaft 150 instead of through basket shaft 150 and basket 140 as illustrated in FIG. 1A, optionally through a separate lumen of a multi-lumen delivery catheter 102.

Referring now to FIG. 1B, collection basket 140^{a} is extended from delivery catheter lumen 104, past the distal end 106 of delivery catheter 102, and expanded within the LPA 456, with embolus deposit opening 142^{a} at its proximal end. While the proximal embolus deposit opening 142^{a} is illustrated in FIG. 1B to face the RPA ostium, it is to be understood that this is shown by way of illustration and not limitation, and that the proximal direction the embolus deposit opening 142^{a} faces depends on the anatomical structure, which can be a Y-shaped or otherwise-shaped anatomy in some cases. When the distal end of apparatus 100^{a} is positioned in the LPA 456, collection basket 140 can be deployed out of delivery catheter 102 by retracting the delivery catheter 102 in a proximal direction, relative to collection basket 140^{a} (which can be optionally kept in position by basket shaft 150), and/or by pushing the collection basket 140^{a} in a distal direction, relative to delivery catheter 102, such as by using basket shaft 150.

According to some examples, collection basket 140, such as collection basket 140^{a}, may include a spring-like mechanism, configured to assume an expanded configuration when in a non-restricted space, and to be compressible into a collapsed configuration when being inserted or passing through a space having a diameter smaller than the diameter of the collection basket in the expanded state. Upon exiting delivery catheter lumen 104, collection basket 140 is released from the restraint induced thereupon by the inner surface of delivery catheter 102 and assumes its expanded configuration. It is to be understood that in such cases, the embolus deposit opening 142, such as embolus deposit opening 142^{a}, can be in a closed state when the collection basket 140 is retained in a compressed state, and to transition to the open state when the basket 140 expands.

In some examples, collection basket 140 is configured to transition between a folded or collapsed configuration, and an expanded configuration. During advancement within the patient's vasculature, collection basket 140 may be in its collapsed configuration, optionally due to external constraint produced by the wall of delivery catheter lumen 104, which prevents collection basket 140 from expanding. Upon removal of the constraint, e.g., when collection basket 140 is extended out of distal end 106 of delivery catheter 102, collection basket 140 may automatically assume its expanded configuration. In some examples, collection basket 140 may be in its folded or collapsed configuration due to at least one condition of the surroundings, e.g., temperature, and may transition to the expanded configuration upon changing of the surrounding condition. In some examples, collection basket 140 can be configured to be controllable shifted between a collapsed configuration and an expanded configuration by an actuation mechanism controllable by the physician, for example by including an umbrella-like arrangement.

Basket 140 may be made, at least partially, of self-expandable metal, such as nickel titanium alloy (Nitinol) or a self-expanding polymer, such as shape memory polyurethane. According to some examples, collection basket 140 is made of a self-expanding braided tube or frame. According to some examples, collection basket 140 is made of a self-expanding frame covered at least partially with wires, mesh, braids, and the like, which form basket walls 146. In some examples, collection basket 140 is not necessarily self-expandable, and may be selectively shifted from a collapsed configuration to an expanded configuration, and vice versa, by an operator, such as a clinician or physician. Such examples may provide enhanced control of the location in which collection basket 140 is expanded and positioned within the target blood vessel.

According to some examples, when in an expanded configuration the broadest portion of collection basket 140, which can be either defined as the expanded diameter of embolus deposit opening 142, or a maximal diameter of the basket 140 defined at any other position between the opening 142 and permanently closed end 143, can have a diameter in the range of about 25%-200% of the diameter of the blood vessel in which it is expanded. In some examples, the maximal diameter of basket 140 in an expanded state thereof is in the range of about 25%-100%, of 25%-75%, of 50%-100%, of 50%-150%, or of 90%-200%, of the diameter of the blood vessel in which it is expanded, including each value within the specified ranges. For example, when collection basket 140 is positioned in the RPA having a diameter of about 18 mm, the maximal basket diameter in its expanded state can be about 4.5 mm, about 6 mm, about 9 mm, about 10.5 mm, about 14.5 mm, about 18 mm, about 15 mm, about 27 mm, or about 36 mm.

Walls 146 of collection basket 140 may be at least partially made of a material which is permeable and/or porous, such as a mesh, braided or weaved mesh, perforated foil, or any other material which may allow blood to flow through the basket when in an expanded configuration, while hindering and preferably preventing the passage of a collected embolus or its fragments therethrough. It is to be understood that any reference throughout the specification and the claims to “walls 146” in the plural form is not meant to be limiting to a plurality of separate walls, and may refer to a single continuous wall that can define one or more portions extending around a circumference of the basket cavity 148.

According to some examples, walls 146 may be include a perforated fabric, metal, polymer, film, foil, or combination thereof. Perforation can be done using laser or mechanical drilling, chemical etching or any other method of perforation known in the art. According to some examples, walls 146 may be made from a net-like fabric, in which yarns are knitted, knotted, looped, or twisted at intersections forming a fabric with open spaces and holes. These perforation or holes of walls 146 may have any geometric shapes, such as square, hexagonal or octagonal. Net fabric may be manufactured using any construction method known in the art, e.g., Tricot, Raschel, and Bobbinet.

According to some examples, walls 146 may be made of fibers such as silk, collagen, fibrin, polyester rayon, Dacron, acetate or nylon, PET, polyurethane, PTFE, polyethylene, UMWPE (ultra-high molecular weight polyethylene), Polyimide, Polyethylether ketone (PEEK), nickel-titanium, stainless steel, Elgiloy, titanium, and/or combinations or derivatives thereof. This composition of walls 146 may determine basket characteristics, such as coarseness and stiffness. The apertures of walls 146 provide filtering functionality, not allowing passage of a collected embolus while at the same time allowing blood flow therethrough.

According to some examples, the open area of walls 146 may be in the range of about 5% to about 99% of the total surface area of walls 146, including each value within the specified range. Open area is defined herein as the portion of the overall surface defined by walls 146 which is not covered by any material. The percentage of open area of walls 146 may affect various functions of collection basket 140, for example, the flexibility of collection basket 140, the rate or ease of blood flow therethrough, the pressure applied on a collected embolus and subsequent fluid removal therefrom when the collection basket 140 is compressed, and more.

According to some examples, the size of the perforations in walls 146 may be in the range of about 1 μm to about 100 μm, of about 50 μm to about 200, of about 100 μm to about 500 μm, of about 200 μm to about 1 mm, or of about 0.5 mm to about 2 mm, including each value within the specified ranges. According to some examples, the thickness of walls 146 may be in the range of about 5 μm to about 50 μm, of about 20 μm to about 100 μm, of about 50 μm to about 250 μm, or of about 100 μm to about 500 μm, including each value within the specified ranges. In some examples, such as when utilized in the PA, the length of the basket 140 can be in the range of about 10 mm to about 180 mm. In some examples, the length of basket 140 (measured between the embolus deposit opening 142 and the permanently closed end 143) is between about 30 mm and about 100 mm.

Rim 144^{a} of collection basket 140^{a} may be formed of a ring which is angled relative to the distal end portion of basket shaft 150^{a}. In some examples, rim 144^{a} is angled at an obtuse angle with the distal end of basket shaft 150^{a} (i.e., a plane defined by the rim 144^{a} is angled at an obtuse angle relative to an axis extending through basket shaft 150^{a}), which can allow for easier re-insertion of basket 140 into delivery catheter 102. In some examples, rim 144^{a} defines an angle in the range of between 110-160 degrees relative to the distal end of basket shaft 150^{a}.

Basket shaft 150 may assist in delivering and maintaining collection basket 140 in position during an embolus capture and collection procedure, and pulling basket 140 back towards distal end 106 of delivery catheter lumen 104. In some examples, basket shaft 150 can be utilized to retract at least a portion of basket 140 into delivery catheter lumen 104, subsequent to placement of embolus 70 in basket cavity 148. In some examples, basket shaft 150 can be utilized to fully retract the basket 140 into delivery catheter lumen 104, subsequent to placement of embolus 70 in basket cavity 148.

Basket shaft 150 may include a flexible wire, a rigid wire, or a tube, and may be made of a polymer, e.g. Pebax, polyurethane, PTFE, polyimide, polyamide, PEEK; or of metal e.g., stainless steel, nitinol, titanium or any other biocompatible material. Basket shaft 150 may be coupled to a handle at a proximal end thereof (not shown), external to the patient, which allows the physician to control the basket's position and/or configuration. Collection basket 140 and basket shaft 150 may be made or be coated with a radiopaque material or include at least one radiopaque marker. In some examples, basket shaft 150 includes a tube that defines a lumen through which a guidewire (e.g., delivery guidewire 152) can be passed. In some examples, basket shaft 150 includes a tube that defines a lumen through which the nosecone shaft 109 extends.

In some examples, the retrieval shaft 120, such as retrieval shaft 120^{a}, can be advanced over retrieval guidewire 128, which can be separate from delivery guidewire 152. Embolus removal apparatus 100 further comprises a capture mechanism 129, configured to capture an embolus 70 and coupled it to a distal end of retrieval shaft 120, keeping it in position during movement of the retrieval shaft 120. In some examples, the capture mechanism 129 comprises an embolus capture element 130 that can be expanded on a distal side of an embolus. In some examples, the retrieval guidewire 128^{a} comprises an embolus capture element 130, for example at a distal portion thereof.

In some examples, retrieval guidewire 128 can be used to facilitate advancement of the retrieval shaft 120 toward a target embolus 70. In some examples, retrieval guidewire 128 can be advanced towards and through a target embolus 70, so as to cross or at least partially penetrate the target embolus 70. For examples, retrieval guidewire 128 is shown to extend through a first embolus 70a located in the RPA. When retrieval guidewire 128^{a} is equipped with an embolus capture element 130, the guidewire 128^{a} can extend through the target embolus 70 so as to position the embolus capture elements 130 within or distally to the embolus 70. For example, an embolus capture element 130 is schematically illustrated to be comprised in a distal portion of the guidewire 128^{a} in FIG. 1B, position distally to the first embolus 70a. The embolus capture element 130 can apply a force against embolus 70, such that upon proximal retraction of guidewire 128^{a} and capture element 130 towards retrieval shaft 120, and/or distal advancement of retrieval shaft 120 towards capture clement 130, the embolus 70 is approximated to the distal end of retrieval shaft 120, optionally until it is sandwiched between the capture element 130 and retrieval shaft 120.

While retrieval guidewire 128^{a} is illustrated in FIGS. 1A-1G to include an embolus capture element 130, it is to be understood that this is shown by way of illustration and not limitation, and that in some examples, a retrieval guidewire 128 can be provided without an embolus capture element 130. In some examples, capture mechanism 129 comprises an embolus capture element 130 that can be carrier on or attached to a separate shaft (not shown) that can extend through retrieval shaft 120, optionally over a retrieval guidewire 128. A separate shaft comprising an embolus capture element 130 can be provided instead of, or in addition to, an embolus capture element 130 attached to a retrieval guidewire 128^{a}. In some examples, the retrieval shaft 120 can comprise an embolus capture element 130. In some examples, apparatus 100 does not necessarily include a retrieval guidewire 128. In some examples, retrieval shaft 120 can be advanced toward a target embolus 70 and/or be maneuvered to carry an embolus 70 into basket 140 without the use of a guidewire extending therethrough.

In some examples, as illustrated throughout FIGS. 1A-1I, a retrieval shaft 120^{a} of the embolus removal apparatus 100^{a} can be designed for advancement over retrieval guidewire 128. In some examples, such as when retrieval guidewire 128 does not necessarily include an embolus capture element 130, the guidewire 128 can be advanced toward target embolus 70, optionally terminating proximate to embolus 70, without necessarily penetrating thereinto, in which case retrieval guidewire 128 is mainly utilized to allow advancement of the retrieval shaft 120^{a} thereover. According to some examples, retrieval guidewire 128 can include an atraumatic distal end, and may further include a hydrophobic coating, a hydrophilic coating, or a lubricant, for casing the passage of the guidewire 128 within the blood vessel (i.e., lowering the friction), and/or through any target embolus 70 without significantly fragmenting it, for enhanced case and safety of delivery.

As shown in FIG. 1C, retrieval shaft 120 can be advanced out of delivery catheter lumen 104, optionally along retrieval guidewire 128, towards a target embolus, such as first embolus 70a. While not explicitly illustrated in FIG. 1C, it is to be understood that in some examples, retrieval shaft 120 can include an atraumatic nosecone, as will be described hereinbelow with reference to FIGS. 10A to 10D. Retrieval shaft 120 includes a distal portion 122 and a main retrieval shaft body 126 proximal to the distal portion 122. The main retrieval shaft body 126 can optionally extend all the way to the handle (not shown), and can define, in some examples, a uniform inner diameter along its length. In some examples, the distal portion 122 can be configured to flare radially outwards in the distal direction. When provided as a flared or tapering distal portion 122, its distal inner diameter is larger than the inner diameter of the main retrieval shaft body 126. In some examples, distal portion 122 may be self-expandable, configured to assume an expanded flared configuration when residing in a non-restricted space, and to be compressible into a collapsed configuration when being inserted into or passed through a space having a diameter smaller than the diameter of its distal end in the expanded state, as will be described in greater detail for distal portion 1221 with respect to FIGS. 9A-9B below.

While illustrated in FIGS. 1A-1I to extend through delivery catheter lumen 104, and be advanced towards the treatment site along with delivery catheter 102, it is to be understood that in some implementations, retrieval shaft 120 can be independently advanced through the patient's vasculature toward the sire of treatment (e.g., toward target embolus 70), without passing through or extending from delivery catheter lumen 104 of delivery catheter 102.

Retrieval shaft 120 is configured to capture and move one or more emboli 70 utilizing at least one capture mechanism 129, as shown in FIG. 1D with respect to a first embolus 70a captured and moved by retrieval shaft 120. As mentioned above, capture mechanism 129 can include, in some examples, an embolus capture element 130. An embolus capture element 130 can be implemented as an elements that can be positioned inside or distally to the embolus 70, such that when the capture element 130 is moved proximally with respect to the embolus 70, it pulls the embolus 70 therewith in the same direction. Examples of embolus capture elements 130 can include, but are not limited to: a hook, a pin, a corkscrew, a stent or other expandable frame, a distal cap, and/or an inflatable balloon.

In some examples, the retrieval shaft 120 can include the capture mechanism 129, or be coupled to a capture mechanism 129. In some examples, the capture mechanism 129 is implemented as suction force applied at the distal end of the retrieval shaft 120. Suction force can be used instead of, or in combination with, one or more embolus capture element(s) 130. Suction force can be generated by inducing a vacuum (the term “vacuum” or “reduced pressure” may be used interchangeably, both indicating a pressure lower than the pressure in the vicinity of target embolus 70) at the tip of distal portion 122, which exerts a pulling force on target embolus 70, as shown for example for first embolus 70A in the illustrated example. Vacuum can be generated through a lumen defined by retrieval shaft 120, which can be in fluid communication with a vacuum generator, typically (but not necessarily) at its proximal end externally to the patient. The vacuum generator may include a syringe, an inflation/deflation device, or any other vacuum generation means, preferably equipped with a locking means to hold the vacuum.

In some examples, the capture mechanism 129 is implemented as a positively charged electrode that applies an electric field configured to attach an embolus 70 thereto. Specifically, an embolus 70 may include charges proteins, enzymes, phospholipids, mineral ions and platelets. The retrieval shaft 120, and specifically, its distal portion 122, can be equipped with two electrodes (not illustrated): an active electrode that can be connected to a terminal power source (for example, outside the patient's body), and a reference electrode connected to another terminal of the power source. When the distal portion 122 with both electrodes is approximated to the embolus 70, voltage applied between both electrodes can anchor the embolus 70 to the active electrode.

In some examples, the capture mechanism 129 is implemented as cryogenic cooling. For example, the distal portion 122 of the retrieval shaft 120 can be cooled to a cryogenic temperature to produce a small frozen adhesion between the embolus 70 and the distal portion 122. This can be achieved by flowing cryogenic fluid through a dedicated lumen of retrieval shaft 120 toward the tip or lip of distal portion 122.

As mentioned above, suction, as well as any of voltage and/or cryogenic cooling, can be implemented as an optional capture mechanism 129 in combination with an embolus capture element 130, optionally comprised in a guidewire 128 as illustrated in FIG. 1C. In such cases, the embolus capture element 130, optionally positioned distal to the embolus 70, can be pulled proximally towards retrieval shaft 120, so as to enhance the coupling of an embolus 70, such as first embolus 70a, to retrieval shaft 120, by exerting mechanical pressure pressing the embolus 70a against the distal portion 122 of retrieval shaft 120. This can be in addition to the suction force applied by the retrieval shaft 120 (or voltage or cryogenic freezing), essentially trapping embolus 70a between capture element 130 and retrieval shaft 120. In some implementations, capture mechanism 129 is implemented as suction force applied to the embolus 70 to pull the embolus towards and against the distal portion 122, and maintain a pull force keeping embolus 70 coupled to distal portion 122 as long as suction is maintained.

According to some examples, the pressure applied to embolus 70, such as first embolus 70a, by a capture mechanism 129 implemented as suction force, may cause embolus 70 to compress, reduce in length, twist, buckle, or otherwise deform. The pressure may also cause embolus 70 to be coupled to and possibly at least partially enter into distal portion 122 of retrieval shaft 120. In some examples, retrieval shaft 120 can further include means (not shown) configured to limit the depth of embolus 70 penetration into retrieval shaft 120. Such means can include any of: a mesh, an orifice, a bump and/or a protrusion in the lumen of retrieval shaft 120, shaped to form a narrowing inside the lumen of retrieval shaft so as to restrict penetration of embolus 70 thereinto. After coupling of the embolus 70 to retrieval shaft 120 is accomplished by any manner described herein, retrieval shaft 120 coupled with embolus 70 can be retracted proximally, removing embolus 70 from the blood vessel in which it was situated.

As further shown in FIG. 1E, retrieval shaft 120 is configured to guide an embolus coupled thereto, such as first embolus 70a, towards and into collection basket cavity 148 through embolus deposit opening 142. In some examples, retrieval guidewire 128 can be reoriented to allow movement of retrieval shaft is a desired direction. In some examples, retrieval shaft 120 can include a curve and/or a steering mechanism, to case the rotation from a first position in which the target embolus 70 was situated, to a second position in which the collection basket 140 is deployed, such as from the RPA 454 to the LPA 456, or vice versa. In such cases, retrieval guidewire 128 can be at least partially retrieved or fully removed from the embolus 70, prior to embolus orientation toward the collecting basket 140, so as to prevent it from interfering with embolus guidance from one position to the other, such as from the RPA 454 to the LPA 456. Once positioned inside collection basket cavity 148, the embolus 70 can be optionally released from the retrieval shaft, such as by no longer applying suction force and/or retracting the embolus capture element 130 out of the embolus.

In the illustrated example, collection basket 140^{a} is deployed within a blood vessel such that collection basket cavity 148 extends from the embolus deposit opening 142 in the downstream (i.e., in the direction of blood flow in the blood vessel the basket is situated in). This may assist in leading the retrieval shaft towards embolus deposit opening 142 and inserting embolus 70 therethrough into collection basket cavity 148. Advantageously, after releasing the embolus 70 from its coupling to retrieval shaft 120 (by releasing the induced vacuum, for example, by terminating the voltage, and/or by elevating the temperature above the cryogenic temperature), the blood flow in such configurations can push embolus 70 towards the distal end of collection basket 140 and prevent embolus 70 or fragments thereof from escaping collection basket 140. This can serve to retain any embolus 70 inside of collection basket cavity 148, with the blood flow pushing the embolus 70 towards the permanently closed end 143^{a} of the basket 140^{a}, without the need to implement active closure mechanisms that will close the embolus deposit opening 142^{a}, as long as the basket 140^{a} retains this downstream orientation.

In some cases, the apparatus 100 can be utilized to capture and place a single embolus 70 inside the basket 140. In such cases, retrieval shaft 120 and any type of capture mechanism 129 can be utilized to capture the embolus 70 and position it within collection basket cavity 148, after which the embolus 70 can be either released from the retrieval shaft 120, or remain attached thereto. Specifically, in some examples, the embolus 70 can be release from retrieval shaft 120, and retrieval shaft 120 can be retracted from the basket 140, allowing further removal of the embolus 70 from the patient's body in a manner similar to that described further below with respect to FIGS. 1H-1I and 5A-5B, for example. However, since the retrieval shaft 120 does not need to be utilized to deliver additional emboli in the case of treating a single embolus 70, it can also remain coupled to the embolus 70, in some examples, such that its main body 126 can extend through the embolus deposit opening 142 even in its closed state, and remain in that configuration during removal of the embolus 70 from the patient's body.

In some examples, the apparatus 100 can be utilized to capture and place a plurality of emboli 70 inside the basket 140, such as first embolus 70a and second embolus 70b in the illustrated example. As shown in FIG. 1F, after releasing the first embolus 70 within collection basket cavity 148, retrieval shaft 120 can be retracted from collection basket 140 and re-advanced towards a second embolus 70b, shown to be situated in the illustrated example in a second branch of the RPA 454. Advancement of the retrieval shaft 120 can be achieved in any manner described above. For example, retrieval guidewire 128 can be directed toward and optionally extended through the length of second embolus 70b, and retrieval shaft 120 can be advanced there-along towards the proximal end of embolus 70b. At least one capture mechanism 129, following any of the examples disclosed herein, can be similarly applied to capture second embolus 70b and couple it to retrieval shaft 120.

As further shown in FIG. 1G, retrieval shaft 120 can be utilized to guide the captured second embolus 70b towards and into the collection basket cavity 148, and as shown in FIG. 1H, the second embolus 70b can be optionally released from the retrieval shaft 120 once positioned within collection basket cavity 148. The apparatus 100 can be utilized for removal of any plurality of emboli 70 from a patient's body. Thus, the process can be similarly repeated to capture any number of additional emboli.

In the example illustrated in FIG. 1H, retrieval shaft 120 is shown to be released from the final second embolus 70b and retracted from collection basket 140, drawn into delivery catheter lumen 104. According to some examples, retrieval shaft 120 may not necessarily disengage from the embolus, and may not be retracted from collection basket 140, such that at least its distal end can remain therein during the withdrawal of embolus removal apparatus 100 from the patient's vasculature.

While the apparatus 100 is demonstrated in FIGS. 1A-1H for retrieval of two emboli, each situated in a different blood vessel, such as a different branch of the RPA, it is to be understood that this is shown by way of illustration and not limitation, and that two or more emboli 70 can be similarly removed from the same blood vessel, such as the same branch of the RPA, by similarly direction the retrieval shaft 120 toward each of the emboli 70 serially situated at different positions in the same branch or blood vessel. Any combination of a plurality of emboli 70, two or more of which are situated in the same branch or blood vessel, and or more additional emboli situated in another branch or blood vessel, are similarly contemplated for removal thereof with embolus removal apparatus 100.

Once all target emboli are placed inside collection basket cavity 148 (including cases of a single embolus or a plurality of emboli), the embolus deposit opening 142 can be transitioned to a closed state, in which escape of the emboli 70 from cavity 148 is prevented during retrieval of the apparatus 100 from the patient's body. It is to be understood that a closed state of the embolus deposit opening 142 does not necessitate full closure or blockage of the opening 142. For example, merely narrowing the embolus deposit opening 142 to a size which still defines an opening through which fluid can flow, but is less than the diameter or maximal cross-sectional size of any embolus 70 deposited in cavity 148, may suffice to serve as a closed state of the opening 142.

In some examples, transitioning of the embolus deposit opening 142 to a closed state is achieved by retracting the embolus removal basket 140 such that at least a portion thereof, including the embolus deposit opening 142, extends into delivery catheter lumen 104. FIG. 1I illustrates collection basket 140, with collected emboli 70a, 70b disposed inside its cavity 148, is retracted proximally, from the LPA 456, towards and at least partially into the distal end 106 of delivery catheter lumen 104. According to some examples, collection basket 140 is retracted such that at least a portion of the collection basket 140 enters through distal end 106 of delivery catheter lumen 104, which may cause rim 144 to compress at least partially.

Continued retraction of collection basket 140 into delivery catheter lumen 104 can advantageously produce a compression force applied by basket walls 146 on the emboli 70 residing therein. As emboli are typically compressible, due to their high liquid content of up to 50%, the compression force may cause the emboli to release at least part of their liquid content, resulting in reduction in the volume and profile of the emboli 70 and, optionally, of collection basket 140. As the embolus diameter may be larger than the inner diameter of delivery catheter 102, possibly significantly so, retrieving collection basket 140 containing the captured emboli may be challenging. Thus, according to some examples and as illustrated, collection basket 140 can be pulled only partially into delivery catheter 102. Even if not retracted fully into delivery catheter 102, the contraction of the embolus deposit opening 142 to a narrower diameter of the delivery catheter lumen 104 can be sufficient to retain the emboli 70 contained within collection basket cavity 148.

In some examples, collection basket 140 remains fully outside delivery catheter lumen 104, in proximity thereto or at a distance therefrom, in which case transitioning the embolus deposit opening 142 to the closed state by a variety of mechanisms described in greater detail below, may be utilized to prevent escape of the emboli from cavity 148. Fully or partially closing the embolus deposit opening 142 can reduce its crossing profile, which can advantageously minimize damage to the surrounding anatomy of the heart or vasculature, during apparatus 100 retrieval, as will be explained in greater detail hereinbelow with respect to FIGS. 5A-5B.

Subsequent to the procedural steps described above with respect to FIGS. 1A-1I, delivery catheter 102 with collection basket 140 at its distal end can be retracted proximally from the pulmonary trunk through the heart right ventricle, the right atrium, and back to the vena cava toward the access site, as further explained below with reference to FIGS. 5A and 5B.

While exemplary implementations of embolus removal apparatus are described and illustrated herein with respect to removing emboli from the pulmonary arteries, such as the LPA, the RPA, and/or branches thereof, it is to be understood that any exemplary embolus removal apparatus disclosed herein can be similarly utilized to remove emboli from any other blood vessel, including at regions of the vasculature that can include bifurcations similar to those shown for the pulmonary arteries, or blood vessels which are not necessarily bifurcated, for example when the embolus removal apparatus is implemented according to some of the examples described below, with respect to FIGS. 6A-8E and others.

Reference is now made to FIGS. 2A-2C, which are schematic illustrations of exemplary embolus removal apparatus 100^{b}, in sequential stages of capturing and delivering an embolus 70 into collection basket 140. Embolus removal apparatus 100^{b} is an exemplary implementation of embolus removal apparatus 100, and thus includes all of the features described for embolus removal apparatus 100 throughout the current disclosure, except that the retrieval shaft 120^{b} is implemented as a tethered retrieval shaft 120^{b}, configured to be pulled into collection basket cavity 148 by pulling on a tether 158 coupled thereto.

Referring to FIG. 2A, retrieval shaft 120^{b} and a collection basket 140^{b} are shown to extend from the distal end of delivery catheter 102. Collection basket 140^{b} can be implemented in a similar manner to that of collection basket 140^{a}, except that it may further include an eyelet 156 as will be described below. Collection basket 140^{b} is expanded within LPA 456, and retrieval shaft 120^{b} is advanced towards embolus 70, shown in the illustrated example to be located within a branch of RPA 454. Retrieval shaft 120^{b} is coupled to a tether 158, such as to an outer surface thereof, at or in the vicinity of distal portion 122. Tether 158 extends from retrieval shaft 120^{b} through embolus deposit opening 142 into basket cavity 148, passes through eyelet 156, and continues into and through delivery catheter lumen 104, wherein the proximal end of tether 158 can be coupled to a pull-and-release mechanism in a handle, or otherwise controlled by a physician externally to the patient.

In some examples, eyelet 156 can be coupled to the inner side of wall 146 of collection basket 140^{b}, i.e., located within basket cavity 148. In some examples, tether 158 is made of a non-stretchable material, such that when a proximal end thereof is pulled (for example, by actuating a knob associated with a tether pulling mechanism in a handle), a pull force is exerted on retrieval shaft 120^{b} towards eyelet 156. According to some examples, tether 158 may be made of a semi-rigid wire or any analogous material, such that also advancing tether 158 by a physician may exert a pushing force on retrieval shaft 120^{b}, optionally assisting advancement of retrieval shaft 120^{b} towards embolus 70. In some examples, retrieval shaft 120^{b} can be pre-shaped to a curved to bent configuration, such that when advanced out of delivery catheter lumen 104, it may assume its curved configuration that may assist in directing it towards embolus 70. In such cases, the tether 158 can be untensioned and include sufficient slack of material to allow the retrieval shaft 120^{b} to assume its pre-shaped configuration, and facilitate uninterrupted advancement thereof toward embolus 70.

Once reaching the embolus 70 in the illustrated example, a capture mechanism 129 that can be implemented according to any of the examples described herein, can be used to couple the embolus 70 to the distal portion 122 of 1205. As illustrated in FIG. 2B, once coupled to embolus 70, retrieval shaft 120^{b} can be drawn towards eyelet 156 within collection basket 140^{b} by pulling tether 158 in a proximal direction. The position of eyelet 156 inside of basket 140^{b} can influence the extent to which the retrieval shaft 120^{b} can be pulled into basket cavity 148. For example, positioning of the eyelet 156 closer to the distal permanently closed end 143 can allow the distal portion 122 to be pulled deeper into basket cavity 148. FIG. 2C illustrates retrieval shaft 120^{b}, together with captured embolus 70, drawn through embolus deposit opening 142 into collection basket cavity 148, by further pulling on tether 158. In some examples, retrieval shaft 120^{b} can be used over a guidewire (e.g., retrieval guidewire 128), and optionally utilize an embolus-capture element 130 for securing the embolus 70.

Reference is now made to FIGS. 3A-3C, which are schematic illustrations of embolus removal apparatus 100^{c}, in sequential stages of expanding a collection basket 140^{c} within a junction of the pulmonary artery, i.e., not necessarily within the confines of the walls of a blood vessel. Embolus removal apparatus 100^{c} is an exemplary implementation of embolus removal apparatus 100, and thus includes all of the features described for embolus removal apparatus 100 throughout the current disclosure, except that the embolus collection basket 140^{c} of embolus removal apparatus 100^{c} comprises a basket shaft 150^{c} that can be implemented as, or include, one or two wire portion 151 that can be used for cinching the rim 144^{c}.

Referring to FIG. 3A, delivery catheter 102 is shown to be positioned in and/or extend through the pulmonary trunk 452, prior to deployment of any of the basket 140^{a} and/or retrieval shaft 120. Collection basket 140^{c} includes rim 144^{c} and wire portion(s) 151, which can be separate components or portions of a continuous single wire. In the illustrated example, a continuous wire is shown to extend along wire portion 151a through delivery catheter lumen 104 towards collection basket 140^{c}, pass through a plurality of hoops at the proximal end of basket walls 146, thereby substantially surrounding embolus deposit opening 142, and then extend away from collection basket 140^{c} along wire portion 151b, with the two wire portions 151 forming a pre-shaped rim 144^{c} therebetween surrounding and intertwined with embolus deposit opening 142. The wire portions 151a, 151b can be spaced apart and parallel to each other.

FIG. 3B illustrates partial deployment of collection basket 140^{c} from the distal end 106 of delivery catheter lumen 104, while FIG. 3C shows the collection basket 140^{c} fully deployed out of delivery catheter lumen 104, free to assume its expanded state within the Pulmonary trunk 452. As illustrated, in some examples, transitioning of the embolus deposit opening 142 to the open state is dictated by rim 144^{c} and basket shaft 150^{c}, by the position of the distal end 106 of delivery catheter 102 prior to exposing basket 140 out of delivery catheter lumen 104, and optionally by the position of the wire portions and the state of their advancement or retraction, and is not necessarily bound by the diameter of a blood vessel in which it is expanded.

In some examples, as illustrated throughout FIG. 3A, the distal end 106 of delivery catheter 102 can be advanced to the position in which the basket 140 should reside when deployed out of delivery catheter lumen 104. As further shown in FIGS. 3B-3C, deployment of basket 140 can be achieved by retracting the delivery catheter 102 relative to the basket 140, while the basket can be kept in position (and/or somewhat distally advanced), such as by utilization of basket shaft 150 to prevent the basket 140 from being retracted along with catheter 102. In some examples, the wire portion(s) 151 can be made of a shape-memory material, as described above with respect to a rim 144 that can be made from a shaped memory material. In such an implementation, the portion of the wire forming the rim 144^{c} can be pre-shaped to assume an expanded diameter of desire when fully deployed. The wire portions 151a, 151b or a shaft that can extend thereover, can be rigid enough to allow axial displacement of the basket 140^{c} thereby, for example to push the basket 140^{c} out of delivery catheter 102, or to retain it in position while delivery catheter 102 is pulled back.

In the configuration illustrated in FIGS. 1A-1B, the rim 144^{a} can be made of a shape-memory material pre-shaped to assume a selected maximal diameter in a free state thereof. However, when expanded in a blood vessel, such as the LPA shown in FIGS. 1A-1B, if the diameter of the blood vessel at the region in which the rim 144^{a} is expanded, is smaller than the maximal diameter the rim 144^{a} can expand to, the actual expanded diameter will be limited to the inner diameter of the blood vessel at this region. In contrast, when the basket 140 is expanded such that the rim 144 is positioned at the level of a junction, between branched vessels, as illustrated in FIG. 3C, the rim 144 may be free to expand to its maximal pre-shaped diameter or to the diameter of the surrounding anatomy, such as the pulmonary trunk diameter, without being bound by outer wall of a smaller blood vessel.

In some implementations, the wire portions 151 can be further utilized as cinching members, such that upon pulling one or both of the wire portions, the wire portion forming the rim 144^{c} is cinched to assume a narrower diameter, thus transitioning the embolus deposit opening to a closed state, in a manner similar to that described in greater detail with respect to FIGS. 4A-4B below.

While optional deployment of a basket 140 such that the rim 144 is positioned at a junction between branched blood vessel, is illustrated in FIGS. 3A-3C with respect to an apparatus 100^{c} that includes a wire forming both the wire portions 151 of basket shaft 150^{c} and the rim 144^{c} of basket 140^{c}, this is not meant to be limiting and shown only for convenience, and it should be understood that any other exemplary basket 140 of any other apparatus 100 disclosed herein can be similarly deployed so as to position the rim 144 at the level of a junction, including, for example, deployment of a basket 140^{a} with a basket shaft 150^{a} formed as a separate component than rim 144^{a}, such as basket 140^{a} illustrated in FIGS. 1A-1I, can be similarly deployed to position the rim 144^{a} at a vascular junction in a manner similar to that shown in FIGS. 3A-3C. Similarly, basket 140^{c} can be deployed in any other portion of the vasculature, including such that the rim 144^{c} is positioned inside a blood vessel or a branch, such as the position in the LPA shown in FIGS. 1A-1I.

FIGS. 4A-4B show the basket 140^{c} described above with respect to FIGS. 3A-3C, in an open state of the embolus deposit opening 142 (FIG. 4A) and a closed state thereof (FIG. 4B). In some examples, the wire portions 151 of basket shaft 150^{c} extend in parallel through a collection basket cover-sheath 154. When the wire portions 151 are not tensioned, the portion forming the rim 144^{c} looped around the circumference of embolus deposit opening 142 can assume it open state, having a diameter wide enough to allow passing of emboli 70 therethrough, into basket cavity 148, as described. FIG. 4A illustrates an embolus 70 positioned inside basket cavity 148, after which, if no more emboli need to be retrieved, the embolus deposit opening 142 can be transitioned to the closed state.

FIG. 4B shows the embolus deposit opening 142 in a closed state, defining a diameter which is less than the cross-sectional size of the embolus 70 trapped in basket cavity 148, preventing it from escaping back to the blood stream therethrough. Transitioning the opening 142 to the closed state can be achieved by pulling on one or both of the wire portions 151, relative to the rim 144^{c}. Thus, cinching of the rim 144^{c} requires one or both wire portions 151 to be pulled while the rim 144^{c} is kept in position and is not axially pulled therewith. For that end, basket shaft 150^{c} can include, in some examples, at least one collection basket cover-sheath 154 is disposed around at least one of the wire portions 151, configured to provide a counter-force acting against the basket 140^{c}, to prevent it from being proximally displaced while one or both of the wire portions 151 is pulled.

In the example illustrated in FIGS. 4A-4B, a single collection basket cover-sheath 154 is shown to extend over both wire portions 151a, 151b, such that both wire portions 151a, 151b can extend, side by side, through a lumen defined by the collection basket cover-sheath 154. The collection basket cover-sheath 154 can extend through the delivery catheter lumen 104, for example all the way to a handle, and can be controlled to remain in position while wire portions 151 are proximally pulled, such that the portion of the wire forming the rim 144^{c} of basket 140^{c} may contact a distal lip 155 of the collection basket cover-sheath 154, preventing the basket 140^{c} from being pulled, such that the axial pulling of the wire portions 151 relative to basket 140^{c} serves to cinch the portion of the wire laced through the proximal end of the basket 140^{c} around the opening 142, resulting in a cinched or closed state shown in FIG. 4B.

In some examples, two collection basket cover-sheath 154 are provided, each disposed around a different one of the two wire portions 151, in which case, one or both wire portions 151 can be pulled, independently or simultaneously, and one or both collection basket cover-sheath 154 can provide the counter force to prevent proximally oriented translation of the basket 140^{c}. In some examples, one collection basket cover-sheath 154 can be disposed only over one of the wire portions 151. In some examples, only a single wire portion 151 is provided, similarly laced or threaded through the proximal portion of basket 140^{c} to form rim 144^{c}, and a single collection basket cover-sheath 154 can be disposed only the single wire portion 151, wherein pulling on the single wire portion 150^{c} while utilizing the collection basket cover-sheath 154 to prevent proximally oriented translation of the basket 140^{c} can similarly cinch the rim 144^{c} and transition the opening 142 to the closed state.

In some examples, the one or more collection basket cover-sheath 154 can be further utilized to cinch the rim 144^{c} by being distally pushed against rim 144^{c}, either without pulling the wire portion(s) 151 but rather keeping them from being similarly translated in the distal direction as well, or while the wire portion(s) 151 are simultaneously pulled. In some examples, the one or more collection basket cover-sheath 154 can be further utilized to control axial displacement of the basket 140^{c}. For example, when distal translation of the basket 140^{c} is desired, such as for the purpose of deployment, the collection basket cover-sheath 154 can be pushed distally, pushing against the rim 144^{c} of the basket and moving it therewith, while the wire portion 151 are allowed to distally move therewith. When retraction of the basket 140^{c} is desired, the wire portion 151 can be proximally pulled to pull the basket 140^{c} therewith, while the collection basket cover-sheath 154 is also simultaneously pulled to allow the proximally-oriented movement of the basket 140^{c}.

FIGS. 5A-5B show exemplary steps in the removal of emboli 70 trapped in the collection basket 140 of embolus removal apparatus 100. FIG. 5A schematically illustrates the embolus removal apparatus 100 extending through the patient's vasculatures, with the distal portion comprising the basket 140 with emboli 70 trapped therein, at a state similar to that described above with respect to FIG. 1I.

During an emboli removal procedure that can include any of the exemplary procedural steps described herein, including the steps described above with respect to FIGS. 1A-3C, and/or steps that will be described below with respect to FIGS. 6A-6D, 10A-10D, and 14, a physician may initially insert a guidewire (e.g., delivery guidewire 152) and/or introducer sheath 110 into a patient's vasculature 410. The introducer sheath 110 may facilitate the subcutaneous insertion of other devices (e.g., guidewires 152 and/or 128, embolus removal apparatus 100 with delivery catheter 102 and basket shaft 150, retrieval shaft 120, etc.) into the patient's vasculature 410, and the guidewire 152 may guide these devices through the patient's vasculature 410 to help prevent them from perforating the walls of the blood vessels and/or other vasculature tissue in route to the target site of treatment, such as main pulmonary artery 450 (or any other blood vessel from which removal of an embolus 70 is desired).

To establish access to the patient's vasculature 410, a physician can make an incision or entry opening 402 (e.g., needle puncture) in a target blood vessel, such as for insertion of an introducer sheath, using any suitable technique, such as the standard Seldinger technique. With the needle inserted into the target blood vessel, the physician can insert delivery guidewire 152 through the needle and into target blood vessel. The physician can remove the needle while leaving at least a portion of the guidewire 152 in the blood vessel. The physician can optionally also deploy a dilator (or a series of dilators) to dilate the blood vessel along a portion extending from the entry opening 402. An introducer sheath 110 can be then inserted into the target blood vessel.

After inserting delivery guidewire 152 and introducer sheath 110, the physician may advance the embolus removal apparatus 100 carrying embolus collection basket 140, through the introducer sheath 110, over delivery guidewire 152, and into the patient's vasculature 410. The physician may continue to advance the embolus removal apparatus 100 through the patient's vasculature 410 until the distal end of apparatus 100 (e.g., distal end 106) is positioned near the site in which an embolus 70 is situated (e.g., a branched pulmonary artery), after which steps may be carried following those described herein, for examples with respect to FIGS. 1A-3C, and/or steps that will be described below with respect to FIGS. 6A-6D, 10A-10D, and 14.

The introducer sheath 110 can be selected based on the target blood vessel, and can have an internal diameter substantially greater than that of the delivery catheter 102, to allow removal of the embolus collection basket 140 with trapped embolus 70 therein, which may be substantially larger, in some cases, than the outer diameter of delivery catheter 102. In some examples, the introducer sheath 110 can be an expandable sheath such as a Solopath™ sheath by Terumo, an iSleeve™ sheath by Boston Scientific, an eSheath by Edwards Lifesciences, or any other expandable design known to those skilled in the art of introducer sheath design. An expandable sheath may allow less trauma to the blood vessel during insertion, and possibly adapt its profile to the delivery catheter 102 profile, to minimize trauma during removal of the delivery catheter 102 with trapped embolus 70.

As mentioned above, while illustrated throughout the Figures and described in various procedural examples herein, to be used for removal of emboli 70 from pulmonary arteries, the exemplary embolus removal apparatuses 100 disclosed throughout the specification can be similarly used for removal of at least one embolus from any other blood vessel. While an entry opening 402 is shown in FIGS. 5A-5B and described herein at the position of a femoral vein, allowing insertion of the introducer sheath 110 into the femoral vein, it is to be understood that this is not meant to be limiting, and that other suitable access points for insertion of the introducer sheath 110 into other target blood vessels is similarly contemplated. For example, an embolus removal apparatus 100 can be utilized to remove an embolus from the neurovascular circulation.

The size of the target blood vessel in which an embolus 70 to be removed is situated, and the vasculature leading thereto, can influence the size of the shafts and catheters, including the size of introducer sheath 110, delivery catheter 102, retrieval shaft 120, and the like. In some examples, such as when an embolus removal apparatus 100 is used for removal of an embolus 70 from the neurovascular circulation, the diameter of the introducer sheath 110 may be in the range of about 3 Fr to about 9 Fr, optionally in the range of about 3 Fr to about 6 Fr, including each value within the specified range. In some examples, such as when an embolus removal apparatus 100 is used to treat deep vein thrombosis, the diameter of the introducer sheath 110 may be in the range of about 6.5 Fr to about 22 Fr, including each value within the specified range. In some examples, such as when an embolus removal apparatus 100 is used for removal of an embolus 70 from a main blood vessel, the diameter of the introducer sheath 110 may be in the range of about 12 Fr to about 40 Fr, optionally in the range of about 12 Fr to about 30 Fr, including each value within the specified range.

In some examples, such as when an embolus removal apparatus 100 is used for removal of an embolus 70 from the pulmonary arteries, the entry opening 402 may be formed in the jugular vein or in the femoral vein, the diameter of the introducer sheath 110 may be in the range of about 10 Fr to about 30 Fr. In the example illustrated in FIG. 5A, the introducer sheath 110 is inserted into the femoral vein 412 in the pelvic area of the patient 400. The embolus removal apparatus 100 is inserted, through the introducer sheath 110, into the femoral vein 412 through the inferior vena cava 416 to the patient's heart 420. It will be understood, however and as mentioned above, that other access locations into the patient's vasculature 410 are possible and are consistent with the present disclosure. For example, when embolus removal apparatus 100 is to be used for treatment of pulmonary embolism, access can be gained surgically via the right ventricular apex, or subcutaneously through the jugular vein, the subclavian vein, the brachial vein or any other vein that connects or eventually leads to the superior vena cava. Use of other blood vessels that are closer to right atrium 422 of the patient's heart 420 may require reduced length of components of the embolus removal apparatus 100 needed to reach the pulmonary embolism.

Returning to the exemplary access path illustrated in FIG. 5A, the embolus removal apparatus 100 can then be guided through the right atrium 422, through the tricuspid valve 458, into the right ventricle 424, through the pulmonary valve 460 into the main pulmonary artery (MPA) 450, optionally positioning the distal end 106 of delivery catheter 102 at the pulmonary trunk 452.

Subsequent to placement of emboli 70 in basket 140, and transitioning the embolus deposit opening 142 to the closed state, as shown in FIGS. 11 and 5A for example, the embolus removal apparatus 100 can be pulled through the patient's vasculature for complete removal thereof, along with emboli 70 carried thereby, from the patient's body, as illustrated in FIG. 5B. In some examples, the introducer sheath 110 can include a funnel or inverted cone-like shaped opening at the distal end of its lumen, to ease the entry and passage therethrough of delivery catheter 102 and collection basket 140, often containing bulky captured emboli 70. Delivery catheter 102 may then be cleaned and reused, or a new delivery catheter 102 may be inserted, to treat additional emboli 70 in the same or other blood vessels.

In some cases, where the shape or size of the captured embolus 70 does not allow its evacuation via introducer sheath 110, the physician may pull collection basket 140 containing the captured emboli 70 close to the distal end of introducer sheath 110 and remove the sheath 110 together with delivery catheter 102 and collection basket 140 from the body. This can advantageously case the removal of large embolus 70 from the body without a need to use or exchange the sheath to a larger, more traumatic introducer sheath. In some cases, a surgical incision may be required for removing a very large embolus 70 captured in collection basket 140. In some examples, following removal of embolus removal apparatus 100 and introducer sheath 110 from the patient's body, delivery guidewire 152 is maintained within the patient's vasculature 410, so as to allow re-access to the treated blood vessel for further treatment, and/or a controlled closure of the access site.

FIGS. 6A-6D are schematic illustrations of an exemplary embolus removal apparatus 100^{d}, in sequential stages of an exemplary embolus removal procedure utilizing apparats 100^{d}. Embolus removal apparatus 100^{d} is an exemplary implementation of embolus removal apparatus 100, and thus includes all of the features described for embolus removal apparatus 100 throughout the current disclosure, except that unlike embolus removal apparatus 100^{a}, the embolus deposit opening 142^{d} of apparatus 100^{d} is at the distal end of collection basket 140^{d} and the permanently closed end 143^{d} is at the proximal end of collection basket 140^{d}. Furthermore, basket shaft 150^{d} can define an internal lumen through which retrieval shaft 120^{d} can extend. The retrieval shaft 120^{d} of apparatus 100^{d} is shown to be extendable through the basket shaft 150^{d} and basket 140^{d}, such that when the retrieval shaft 120^{d} extends distally to the basket 140^{d}, such as past embolus deposit opening 142^{d} in the open state of opening 142^{d}, the retrieval shaft 120^{d} extends from the basket shaft 150d, through the permanently closed end 143^{d}, toward and through the embolus deposit opening 142^{d}.

During delivery, both the retrieval shaft 120^{d} and basket shaft 150^{d} with basket 140^{d} attached thereto, can be disposed within delivery catheter lumen 104, with basket 140^{d} in a collapsed or compressed state, and can be optionally concentric to each other. The basket shaft 150^{d} can be attached to the permanently closed end 143^{d}. FIGS. 6A-6D shows stages of an exemplary method utilizing embolus removal apparatus 100^{d} for removal of two emboli 70 shown to be disposed at two branches of the RPA. FIG. 6A illustrates the delivery catheter 102 located within a pulmonary trunk, with collection basket 140^{d} deployed out of delivery catheter lumen 104, such as by axially pushing the basket shaft 150^{d} relative to delivery catheter 102, and/or proximally pulling the delivery catheter 102 relative to the basket shaft 150^{d}.

In some examples, components of the embolus removal apparatus 100^{d}, such as delivery catheter 102, basket shaft 150, and retrieval shaft 120^{d}, are axially movable to each other, together forming a telescopic-like structure, such that retrieval shaft 120^{d} is axially movable through and relative to a lumen of the basket shaft 150^{d}, and basket shaft 150^{d} is axially movable through and relative to delivery catheter lumen 104. In some examples, such as when retrieval shaft 120^{d} of apparatus 100^{d} extends through basket shaft 150^{d}, the retrieval guidewires 128 passing through a lumen of retrieval shaft 120^{d} also serves as the delivery guidewire (i.e., instead of separate guidewire 152), over which embolus removal apparatus 100^{d} can be advanced through the patient's vasculature.

When deployed out of delivery catheter lumen 104, collection basket 140^{d} may be free to expand, thereby transitioning the embolus deposit opening 142^{d} to the open state. This can be achieved, for example, by forming the collection basket 140^{d} or suitable portions thereof from shape-memory material that can be pre-shaped to self-expand, as described above, or by including a suitable self-expanding structure, such as a mesh tube (that can be optionally made of stainless steel or other suitable materials) configured to expand to a pre-formed diameter. Deployment of the collection basket 140^{d} can be performed such that the embolus deposit opening 142^{d} is facing the target embolus 70 for removal. In the illustrated example, basket 140^{d} is expanded in the RPA such that the embolus deposit opening 142^{d} is facing the blood vessels branched from the RPA and including the emboli 70.

As further shown in FIG. 6A, with the embolus deposit opening 142^{d} in the open state, retrieval shaft 120^{d} can be navigated towards and may engage and capture embolus 70, such as first embolus 70a, according to any manner described above, including employing suction as an optional capture mechanism 129. Once the embolus 70a is coupled to the distal portion 122, retrieval shaft 120^{d} can be proximally retracted, passing through embolus deposit opening 142^{d}, until the embolus 70a is positioned within collection basket cavity 148, at which point it can be release from retrieval shaft 120^{d} as shown in FIG. 6B.

In some examples, collection basket 140^{d} can extend into the right ventricle (not shown in FIGS. 6A-6D). In some examples, only collection basket 140^{d} can be initially at least partially exposed, in a manner that allows it to expand inside the pulmonary trunk but not across the pulmonary valve at this initial stage. In such cases, as an embolus 70 pulled back by retrieval shaft 120^{d} for example, into the basket 140^{d}, the remaining length of the basket 140^{d} may be allowed to expand, extending through the pulmonic valve into the right ventricle. Minimizing the time it takes to place a relatively large basket 140 across the pulmonic valve can be of utmost importance, in some cases, for the patient's hemodynamic stability. In some examples, basket 140^{d} can be simultaneously expanded during proximal pull back of the retrieval shaft 120^{d} and the embolus 70 coupled thereto. For example, a distal portion of the basket 140^{d} can extend into the LPA, the RPA, or the main pulmonary artery, while its proximal portion extends into the right ventricle. Upon proximal pull-back of the retrieval shaft 1204, basket 140^{d} can be expanded, including the proximal portion of the basket extending into the right ventricle.

In some examples, collection basket 140 can be advanced distally while embolus 70a is coupled to the distal portion 122, until embolus 70a is positioned within collection basket cavity 148. As shown, the basket 140^{d} can narrow in diameter from the embolus deposit opening 142^{d} to the permanently closed end 143^{d}, such that the narrow end between the porous basket walls 146 at the permanently closed end 143^{d} and any shaft passing therethrough, such as retrieval shaft 120^{d}, is too narrow to allow further displacement of the embolus 70 proximal to permanently closed end 143^{d}. In some examples, the permanently closed end 143^{d} can be sized to define a diameter similar to that of the basket shaft 150^{d} it is attached to, to allow integration therewith.

As the direction of the blood flow in the illustrated example is from the proximal end at of basket 140^{d} at the permanently closed end 143^{d} to the distal end at the embolus deposit opening 142^{d}, insertion of embolus 70 into collection basket 140^{d} is performed against the direction of blood flow, posing a continuous risk of embolus 70 or any fragment thereof being carried out of basket 140^{d} through embolus deposit opening 142^{d} by the blood flow. To mitigate this risk and retain embolus 70, such as first embolus 70^{a} in the illustrated example, within basket 140^{d}, collection basket 140^{d} can further include, in some examples, a closing mechanism 160 configured to transition the embolus deposit opening 142^{d} to a closed state, even while the basket 140^{d} is fully deployed and expanded out of delivery catheter 102. Transitioning the embolus deposit opening 142^{d} to the closed state by the closing mechanism 160, while collection basket 140^{d} is still expanded can advantageously obstruct at least a portion of embolus deposit opening 142^{d} to hinder passage of emboli 70 therethrough, optionally while still allowing free passage of blood fluid through the embolus deposit opening 142^{d} in its closed state.

Several exemplary implementations of a closing mechanism 160 will be described in greater detail below, such as with respect to FIGS. 7A-8E. In FIGS. 6A-6D, the closing mechanism 160 is schematically illustrated to include cover flaps 164. However, it is to be understood that any other closing mechanism 160 described herein, can be utilized with respect to FIGS. 6A-6D. In some examples, closing mechanism 160 is designed to transition the embolus deposit opening 142^{d} between the open and closed states, such that in the closed state, a narrow opening 162 is maintained, sized to allow passage of retrieval shaft 120 therethrough, even when the embolus deposit opening 142 is in the closed state, while preventing escape of any emboli 70 contained in basket cavity 148 from escaping therefrom. In some examples, basket 140 can include a basket covering 141 that restricts mobility of embolus 70 due to blood flow, as will be explained in greater detail below with respect to FIG. 7B.

As further illustrated in FIG. 6B, the retrieval shaft 120^{d} can be utilized to retrieve additional emboli if required, such as second embolus 70b, as described in detail above with respect to FIGS. 1F-1G for example. Once positioning of all emboli 70 in basket cavity 148 is complete, as shown in FIG. 6C for emboli 70a, 70b, the retrieval shaft 120^{d} can be retracted in any manner similar to those described above with respect to FIG. 1H for example, and the basket shaft 150^{d} can be retracted into delivery catheter lumen 104 as shown in FIG. 6D, optionally such that a proximal portion of basket 140^{d}, including permanently closed end 143^{d} and maybe a portion extending distally therefrom, is placed and may assume a somewhat narrower diameter inside delivery catheter 102, or it can be fully retracted into delivery catheter 102, and optionally squeeze emboli 70 trapped in basket 140^{d}, according to any example described above with respect to FIG. 1I for example.

As shown in FIGS. 6D, emboli 70 remain trapped within basket cavity 148 between the permanently closed end 143^{d} at the proximal end of basket 140^{d}, and embolus deposit opening 142^{d} retained in a closed state by closing mechanism 160 at the distal end of basket 140^{d}. Further retraction of embolus removal apparatus 100^{d} to remove emboli 70 trapped thereby from the patient's body, subsequent to the steps described above with respect to FIGS. 6A-6D, can be performed according to any example described above with respect to FIGS. 5A-5B.

A closing mechanism 160 can function as a one-way valve with respect to emboli 70, allowing embolus 70 coupled to retrieval shaft 120 to be inserted into basket cavity 148 through embolus deposit opening 142, while hindering passage of the embolus 70 outwards from basket cavity 148. While a closing mechanism 160 is illustrated, for example, in combination with basket 140^{d} of apparatus 100^{d} in FIGS. 6A-6D, it is to be understood that a closing mechanism 160 can be similarly utilized or added to any other basket 140 disclosed herein, including any of baskets 140^{a}, 140^{b}, and 140^{c} described above with respect to FIG. 1A-4B. That is to say, a closing mechanism 160 can be added also to collection baskets 140 intended for deployment in blood vessels, such that the baskets 140 are oriented in a downstream direction. This may be useful, for example, for allowing navigation of the collection basket 140 between different positions within the blood vessels, without the necessity of always maintaining the embolus deposit opening 142 in an upstream position relative to basket cavity 148.

In some examples, the closing mechanism 160 is configured to allow selective transitioning of the embolus deposit opening 142 between an open state and a closed state by an operator. In some examples, the closing mechanism 160 is biased to a closed state of the embolus deposit opening 142, and can transition to an open state by movement or altered position of retrieval shaft 120 or of any other component of the apparatus 100. In some examples, the closing mechanism 160 is configured to be fixedly maintained in a substantially closed state.

FIGS. 7A-7D and 8A-8E illustrate some exemplary collection basket 140 equipped with exemplary closing mechanisms 160. It is to be understood that any of the basket 140 described below with respect to FIGS. 7A-8E can be employed in lieu of basket 140^{d} described above with respect to FIGS. 6A-6D, or in lieu of baskets 140^{a}, 140^{b}, or 140^{c} described above with respect to FIGS. 1A-4B, when implemented to include a closing mechanism 160. FIG. 7A shows an exemplary basket 140^{e} comprising a closing mechanism 160^{e} that includes cover flaps 164 overlaying the embolus deposit opening 142^{e}. While three cover flaps 164 are illustrated in FIG. 7A, it is to be understood that any other number of flaps 164 is contemplated, such as a single flap, two flaps, or more than three flaps.

Cover flaps 164 are coupled to rim 144^{e} of embolus deposit opening 142^{e}, and extend towards the center of embolus deposit opening 142^{e}. Cover flaps 164 cover embolus deposit opening 142^{e} in a substantially continuous manner, i.e., such that each flap 164 is in close proximity to its neighboring flap 164, and the flaps 164 can be optionally connected to substantial portion or whole of the circumference of rim 144^{e}. Cover flaps 164 can have any suitable shape configured to allow movement thereof toward and away from each other in a manner that can transition the embolus deposit opening 142^{e} between its open and closed states.

Cover flaps 164 taper from rim 144^{e} towards the center of embolus deposit opening 142^{e}, such that the flaps 164 cover the majority or a substantial area of embolus deposit opening 142^{e}, for example when biased to a closed state of the opening 142^{e} illustrated in FIG. 7A. According to some examples, the tapering flaps 164 do not extend all the way to the center of embolus deposit opening 142^{e} so as to contact each other, to define a narrow opening 162 therebetween, even in the closed state of the opening 142^{e} illustrated in FIG. 7A. Narrow opening 162 can be defined at the center of embolus deposit opening 142^{e} as illustrated, or at any other part thereof. In some examples, narrow opening 162 is sized to allow passage of a retrieval guidewire 128 therethrough. In some examples, narrow opening 162 is sized to allow passage of a retrieval shaft 120 therethrough.

Cover flaps 164 can be configured to be in a closed configuration when not subjected to external pressure, and be pushed to extend away from each other and assume an open configuration when a pressure is exerted thereon in a proximal direction, i.e., towards internal cavity 148 of collection basket 140^{e}. Cover flaps 164 may be maintained in a closed configuration, for example, by springs 166 disposed between flaps 164 and wall 146 of collection basket 140^{e} and/or rim 144^{e}, such that when a pressure is exerted on cover flap 164 in the proximal direction, spring 166 may be compresses if implemented as a compression spring, or deflected if implemented as a leaf spring, and when the pressure is relieved, spring 166 is free to assume its free state to bias the cover flap 164 coupled thereto to the closed configuration.

As mentioned above, spring 166 can be implemented as a coil spring, a leaf spring, or any other suitable type of spring. Other methods of biasing cover flap 164 to a closed configuration may be used as known in the art. In the open configuration, at least a portion of at least one of the cover flaps 164 is pressed to assume a more proximal and/or outwardly oriented position than in the closed configuration. An outwardly oriented direction is the direction from a central axis of the embolus deposit opening 142^{e} towards wall 146 of collection basket 140^{e}. Cover flaps 164 can be pushed into an open configuration by retraction of retrieval shaft 120 into collection basket 140^{e}, optionally, but no necessarily, when coupled to an embolus 70. The number of cover flaps 164 and the degree to which they can be proximally and/or outwardly deflected, when being pushed into an open configuration, may be dynamic and influenced by several factors, such as: the tension of springs 166, the size of the portion of retrieval shaft 120 passing through embolus deposit opening 142^{e}, the size of captured embolus 70, the area of embolus deposit opening 142^{e} which is being covered by cover flaps 164, the material from which cover flaps 164 are made, and the like.

In some examples, the cover flaps 164 can be designed to be mostly spread, in the closed configuration, within the plane defined by rim 144^{e}. In some examples, cover flaps 164 are designed, in the closed configuration, to be angled relative to the plane defined by rim 144^{e}, at an angle in range of about 0° to about 45°, of about 0° to about 30°, or of about 5° to about 15°, including any value within the specified ranges. The angle can be defined in the proximal direction, i.e., such that flaps 164 extend toward basket cavity 148. Nevertheless, in some examples, the angle can be oriented in the distal direction, such that cover flaps 164 extend away from cavity 148.

FIG. 7B shows an exemplary collection basket 140^{f} comprising a closing mechanism 160^{f} that includes a drawstring 168 extending axially along collection basket 140^{f} and coupled to rim 144^{f}, configured to cinch the 144^{f} so as to constrict the size of embolus deposit opening 142^{e} when the drawstring 168 is proximally pulled. The drawstring 168 can be in the form of a wire, suture, string, cable, and the like, extending along the length of basket shaft 150, optionally, but not necessarily, through a lumen basket shaft 150. The drawstring is coupled around a circumference surrounding the embolus deposit opening 142^{e}, such as rim 144^{f}. For example, the drawstring can pass through a plurality of hoops at the rim 144^{f}. The drawstring 168 can extend through a lumen of a drawstring cover shaft 170 extending therearound and terminating proximate to rim 144^{f}.

The rim 144^{f} can include or be formed of a shape-memory material, pre-shaped to assume an expanded free state in which the embolus deposit opening 142^{e} assumes its open state. The drawstring 168 can extend along a length of embolus removal apparatus 100 such as into a handle, and be operable by a physician such that upon pulling of the drawstring 168, the rim 144^{f} is cinched, transitioning the embolus deposit opening 142^{e} to the closed state by constricting it, optionally not entirely but up to a narrow opening 162 as defined above. The drawstring cover shaft 170 can be used to provide a counterforce against the rim 144^{f} during pulling of the drawstring 168. When the drawstring 168 is released and no longer proximally pulled, the rim 144^{f} can be released to assume its free state diameter, transitioning the embolus deposit opening 142^{e} back to the open state. In some examples, blood stream in collection basket 140 may be directed or diverted to a restricted area of the embolus deposit opening 142. Deposited embolus 70 may be thus directed, after being released in the collection basket 140, to a restricted area configured to keep the embolus 70 or fragments thereof from leaving the basket 140. Blood flow may be directed to the restricted area by partially sealing parts of the basket walls 146 or adding a diverter (not shown) inside basket 140 configured to divert flow toward the restricted area of the deposit opening.

In some examples, collection basket 140 further comprises a basket cover 141 extending distally from closed end 143 to a length Lc, which is less than the entire basket length Lb as illustrated in FIG. 7B. Basket cover 141 covers the apertures of basket walls 146, thus restricting blood flow through a portion of the basket 140 along the length Lc. In some examples, Lc is equal to or less than 50% of Lb. In some examples, Lc is equal to or less than 30% of Lb. In some examples, Lc is equal to or less than 20% of Lb. An embolus 70 residing within basket cavity 148 covered by basket cover 141 will be minimally affected by blood flow, which in turn will help maintain the embolus 70 in position. Due to the tapering or conical profile of basket 140, blood can freely flow around the covered portion of the basket 140, preventing the basket covering 141 from fully occluding the vessel blood flow. Basket cover 141 can be formed, in some examples, from graft covering materials known in the art, can be implemented as a coated layer around basket walls 146, or can be implemented as a fine-weaved mesh structured formed around the basket walls 146 along length Lc.

It is to be understood that basket covering 141 is shown in FIG. 7B in combination with a specific type of exemplary collection basket 140^{f} that includes a drawstring 168 by way of illustration and not limitation, and that any other basket 140 disclosed herein, and in particular, any other type of closing mechanism 160, can be implemented so as to include basket covering 141. Similarly, collection basket 140^{f} that includes a drawstring 168 is not necessarily meant to include a basket covering 141, but may be rather implemented with or without basket covering 141.

FIG. 7C shows an exemplary collection basket 140^{g} that does not necessarily includes a closing mechanism configured to transition the embolus deposit opening 142^{g} between open and closed states. The collection basket 140^{g} is rather designed, in its expanded state shown in FIG. 7C, to include an inverted neck 172 of the basket's wall 146 configured to create a region in which embolus 70 inserted into cavity 148 may remain trapped therein. As illustrated, the wall 146 of collection basket 140^{g} extends from the permanently closed end 143^{a} to a widest end portion 174 of the basket 140^{g}, opposite to the permanently closed end 143^{g}, and is then further inverted inwards, such as towards permanently closed end 143^{g} and away from a widest end portion 174, forming an inverted neck 172 that extends radially inward and away from a widest end portion 174.

The inverted neck 172 terminates at the embolus deposit opening 142^{g}, that is narrower than the widest end portion 174. Thus, in this design on collection basket 140^{g}, the embolus deposit opening 142^{g} is not shown to define the widest diameter of the collection basket 140^{g}, and is not position at an end of the basket 140^{g}, but rather at a position between the proximal and distal end of the basket 140^{g}. In some examples, the embolus deposit opening 142^{g} is not configured to change in diameter when the basket 140^{g} is in its expanded state, and is sized to allow extension of a retrieval shaft 120, optionally with an embolus 70 coupled thereto, through the opening 142^{g}.

When retrieval shaft 120 is retracted through the embolus deposit opening 142^{g} to position and release an embolus 70 inside basket cavity 148, the released embolus 70 may tend to move sideways and toward widest end portion 174. In some examples, the retrieval shaft 120, while still coupled to the embolus 70 within basket cavity 148, may be steered inside cavity 148 to direct the embolus sideways, into a portion of cavity 148 bound between inverted neck 172 and wall 146 closed to the widest end portion 174, and release the embolus 70 therein. An embolus 70 such positioned may be trapped in this position even after being released, particularly if the downstream direction is aligned with the direction of the basket 140^{g} from the permanently closed end 143^{g} to the widest end portion 174.

In some examples, collection basket 140^{g} of the type shown in FIG. 7C and described above, can further include a closing mechanism 160^{g} in which the inverted neck 172 is made of a stretchable material, such that a retrieval shaft 120 with an embolus 70 coupled thereto can cause the opening 142^{g} to expand as the neck 172 is stretched, and revert back to a narrower diameter thereafter.

FIGS. 8A-8E show an exemplary basket 140^{h} comprising a closing mechanism 160^{h} that includes a hinged lid 176 covering embolus deposit opening 142^{h}. Collection basket 140^{h} can be coupled at its permanently closed end 143^{g} to basket shaft 150. In some examples, Lid spring 178 can disposed between hinged lid 176 and wall 146 of basket 140^{h} or rim 144^{h}, configured to bias and maintain the hinged lid 176 in a closed position, as illustrated in FIG. 8A. In some examples, actuation wire 180 is connected at a distal end thereof to hinged lid 176, and extends proximally therefrom, such as through the basket cavity 148 and the lumen of basket shaft 150, optionally to proximal end that can be coupled to a handle, operable by a physician to pull, release, and/or push the actuation wire 180. Actuation wire 180 can be in the form of a wire, suture, string, cable, and the like.

Hinged lid 176 is pivotable along hinge 182 with respect to rim 144^{h}, between a closed configuration, in which the lid 176 can be substantially aligned with a plane defined by the 144^{h}, and an open configuration, in which the lid 176 is angled with respect to the plane defined by the 144^{h}. As mentioned, in some examples, lid spring 178, which can be a coil spring, leaf spring, or any other biasing member known in the art, can be set to bias the hinged lid 176 to a closed configuration. In some examples, lid spring 178 is an integral part of the structure or weave that forms lid 176 or rim 144^{h}. Pulling on actuation wire 180 may pivot hinged lid 176 about hinge 182, into the basket cavity 148, transitioning it into the open configuration, as illustrated in FIG. 8C. Releasing the actuation wire 180 allows the hinged lid 176 to revert to its closed configuration, optionally trapping an embolus 70 in cavity 148, as illustrated in FIG. 8D. In some examples, the hinge 182 is also the lid spring 178. In some examples, actuation wire 180 can be rigid enough to allow it to be pushed by the physician, so as to actively transition the lid 176 from an open to a closed configuration. In such examples, the basket 140^{h} can be optionally devoid of lid springs 178, such that it is not necessarily biased to any state, and the transition between the open and closed states can be entirely controlled by pulling or pushing the actuation wire 180.

In some examples, hinged lid 176 includes narrow opening 162, through which at least a portion of retrieval shaft 120 can be advanced and retracted, such as main retrieval shaft body 126. Narrow opening 162 can be further sized to allows passage of an optionally flared distal portion 122 of retrieval shaft 120 therethrough. In some examples, narrow opening 162 is sized to have a smaller diameter than an expected size of an embolus 70, to prevent escape of a collected embolus 70 therethrough. Accordingly, when retrieval shaft 120, with a captured embolus 70 coupled thereto, is retracted, at least one of distal portion 122 and embolus 70 may push proximally against hinged lid 176, causing hinged lid 176 to pivot into an open configuration. In some examples, actuation wire 180 can be coupled to retrieval shaft 120, such that when retrieval shaft 120 is retracted, actuation wire 180 is also pulled back, simultaneously opening lid 176 with shaft 120 retraction. In some examples, the physician can actively pull actuation wire 180 in coordination with retraction of retrieval shaft 120, to facilitate its entry into collection basket 140^{h}.

In some examples, hinged lid 176 does not necessarily define a narrow opening 162, in which case advancement and retraction of retrieval shaft 120 past hinged lid 176 may be achieved by pulling the actuation wire 180 sufficiently to allow such passage of the retrieval shaft 120.

In some examples, the actuation wire 180 is connected to a portion of lid 176 proximate narrow opening 162. In some examples, the actuation wire 180 is connected to a portion of lid 176 at an opposite end of lid 176 relative to hinge 182. FIG. 8B is a top view of an exemplary implementation of collection basket 140^{h}. In some examples, a single actuation wire 180 is provided, as shown in FIG. 8A. In some examples, more than one actuation wire 180 can be provided, such as three actuation wires 180 shown in FIG. 8B to be connected to the lid 176 at different points along a perimeter of narrow opening 162.

As mentioned above and illustrated in FIG. 8D, hinged lid 176 is allowed to revert to its naturally-biased closed configuration, or is actively closed by the operator, such that embolus 70 can be safely released from retrieval shaft 120 within cavity 148, trapping the embolus 70 in cavity 148. Depending on the orientation of basket 140^{h} relative to the direction of blood flow, blood flow can, in some cases, push the embolus 70 towards and against the lid 176. If only a single embolus 70 needs to be removed from the patient's body, the distal portion 122 of retrieval shaft 120 can either remain in cavity 148, or be further retracted into the lumen of basket shaft 150, and the embolus removal apparatus 100 can be removed from the patient's body, along with embolus 70 trapped in basket 140^{h}, according to any example described above.

If an additional embolus 70 needs to be removed, the physician may pull the actuation wire 180 to at least partially open the hinged lid 176, and navigate retrieval shaft 120 (and possibly guidewire 128) out of collection basket 140^{h} and towards the additional embolus, as illustrated in FIG. 8E. the angular pivotable movement of lid 176 during its transition to the open configuration can generally push against the embolus 70 and force it to a region spaced between the lid 176 and the basket walls 146, such that embolus 70 will remain trapped therebetween even when the lid 176 is re-opened. In some cases, the physician can maneuver the retrieval shaft 120 to push embolus 70 away from narrow opening 162, prior to opening lid 176. In some examples, when the distal portion 122 is narrower than narrow opening 162, retrieval shaft 120 can be optionally advanced out of collection basket 140^{h} without having to open lid 176.

An additional embolus 70 can be captured and delivered by the retrieval shaft 120 into the basket cavity 148 in the same manner described above. When all emboli 70 are collected and trapped in basket cavity 148, the distal portion 122 of retrieval shaft 120 can either remain in cavity 148, or be further retracted into the lumen of basket shaft 150, and the embolus removal apparatus 100 can be removed from the patient's body, along with embolus 70 trapped in basket 140^{h}, according to any example described above.

In some examples, elements of collection basket 140 disposed over embolus deposit opening 142, such as cover flaps 164 or hinged lid 176, may be formed of a filtering material, having pores which allow blood to flow therethrough with minimal hindrance, while preventing larger particles, such as emboli 70 or fragment thereof, from passing therethrough, In some examples, cover flaps 164 or hinged lid 176 can be formed to include apertures defining maximal diameters of about 10μ, 20μ, 30μ, 50μ, 80μ, 100μ, 200μ, 350μ, 500μ, 750μ, 1000μ, 1500μ, 2500μ, or 5000μ.

In some examples, distal portion 122 of retrieval shaft 120 is not necessarily flared outwards, but is rather formed to have a uniform diameter with the remainder of retrieval shaft 120, such as having the same diameter as that of main retrieval shaft body 126. In some examples, the distal portion 122 can be narrowed to a smaller diameter than that of main retrieval shaft body 126. In some cases, it may be advantageous to utilize a retrieval shaft 120 having a narrow distal portion (not shown), for example in order to produce a focused suction force on the removed embolus 70 or when the capture mechanism 129 is not implemented to include application of a suction force. According to some examples, the distal portion 122 of the retrieval shaft 120 can be shaped like a nosecone with a distally tapered tip (not shown).

In some examples, distal portion 122 is formed to have an outwardly flared conical or frustoconical shape. In some examples, distal portion 122 is an expandable portion 122 that can transition between a collapsed configuration and an expanded configuration, as demonstrated, for example, for expandable distal portion 122ⁱ in FIGS. 9A-9B. An expandable distal portion 122ⁱ of retrieval shaft 120ⁱ can be retained in a collapsed configuration during delivery to the site of treatment, to occupy less space while retained in delivery catheter 102, allowing for a more compact profile of the embolus removal apparatus 100 as it is passed through narrower blood vessels along the patient's vasculature.

In some examples, a cover-sheath 124 can be disposed over retrieval shaft 120ⁱ and its distal portion 122ⁱ. The cover-sheath 124 and the retrieval shaft 120ⁱ are axially movable relative to each other. FIG. 9A shows the cover-sheath 124 disposed over and around distal portion 122ⁱ, wherein the inner diameter of cover-sheath 124 can be similar to the outer diameter of a non-expanded or non-flared portion of the retrieval shaft 120ⁱ, such as the outer diameter of main retrieval shaft body 126. Thus, in the configuration shown in FIG. 9A, the cover-sheath 124 retains the distal portion 122ⁱ in a compacted configuration.

When approximating the site of treatment and prior to or during advancement towards embolus 70, the distal portion 122ⁱ can be deployed out of cover-sheath 124 as shown in FIG. 9B, allowing it to assume a self-expandable flared configuration. This can be achieved by proximally retracting the cover-sheath 124 relative to retrieval shaft 120ⁱ, and/or distally pushing the retrieval shaft 120ⁱ relative to cover-sheath 124. In some examples, a series of resilient ribs 184 can be located on, attached to, or embedded in, the sides of distal portion 122ⁱ to force it to the expanded flared shape when not bound by a cover-sheath 124. Retrieval shaft 120ⁱ can be used with any of the exemplary embolus removal apparatuses 100 disclosed herein. In some examples, distal portion 122ⁱ is pre-formed in its flared shape and is made of an clastic, semi-elastic, or a shape memory polymer, configured to collapse, fold and/or stretch when inserted into cover-sheath 124, and to substantially regain its original flared shape upon being released therefrom. Exemplary elastic, semi-clastic, or shape memory materials from which distal flared portion 122ⁱ can be formed, can include, but are not limited to: silicone, polyurethane, latex, polyethylene terephthalate (PET), polyethyleneoxide (PEO), any combination thereof and/or other suitable polymers known in the art.

FIGS. 10A-10D are schematic illustrations of an exemplary embolus removal apparatus 100ⁱ, in sequential stages of an exemplary embolus removal procedure utilizing apparats 100ⁱ. Embolus removal apparatus 100 is an exemplary implementation of embolus removal apparatus 100, and thus includes all of the features described for embolus removal apparatus 100 throughout the current disclosure, except that embolus removal apparatus 100^{j} further comprises an expandable nosecone 190. As illustrated in FIG. 10A, embolus removal apparatus 100ⁱ can have a retrieval shaft 120^{j} extendable through a lumen of basket shaft 150 and through basket 140^{j} in a similar manner to that described above for embolus removal apparatus 100^{d} with respect to FIGS. 6A-6D.

In the state illustrated in FIG. 10A, the collection basket 140^{j} is shown to be in a collapsed configuration inside of delivery catheter lumen 104, while the distal portion 1221 of retrieval shaft 120^{j} is shown to be positioned past the basket 140^{j} and the distal end 106 of delivery catheter 102. Retrieval guidewire 128 is further illustrated to have been extended through an embolus 70. In some examples, as illustrated in FIGS. 10A-10D, the distal portion 122^{j} of retrieval shaft 120ⁱ can have a flared portion 186 expanding radially outwards, and a cylindrical portion 188 extending distally from the flared portion 186, the cylindrical portion 188 having a uniform diameter equal to the larger diameter at the distal end of the flared portion 186. In some examples, the distal portion 1221 of retrieval shaft 120ⁱ can be foldable and transition between a collapsed configuration and an expanded configuration, in a similar manner to that described for distal portion 122ⁱ with respect to FIGS. 9A-9B.

It is to be understood that a distal portion 122 having a flared portion 186 and a cylindrical portion 188 can be utilized as the distal portion 122 of retrieval shaft 120 utilized in any of the exemplary embolus removal apparatuses 100 disclosed herein, and that the distal portion 122 of a retrieval shaft 120 used in combination with expandable nosecone 190 does not have to include a flared portion 186 and a cylindrical portion 188, but may rather be implemented as any other exemplary distal portion 122 disclosed herein.

The maximal diameter of the distal portion 122, whenever implemented to flare outwards, such as any non-expandable flared distal portion 122 disclosed herein, expandable distal portion 122ⁱ, or flared portion 122^{j}, can define a maximal diameter which is greater than the diameter of main retrieval shaft body 126 by at least 10%, 20%, 30%, 50%, 75%, 100%, 150%, 200% or 400%. The larger diameter of distal portion 122 in such examples can improve the coupling of an embolus 70 thereto, particularly when the capture mechanism 129 is implemented as suction force applied through the distal portion 122, such that a larger contact area of distal portion 122 with embolus 70 can increase the pulling force exerted on embolus 70. A larger contact area may also enhance the stability of embolus 70 during the retrieval process, reducing the risk of embolus fragmentation. Moreover, this increased suction force can partially pump a portion of the embolus 70 into a conical portion of distal portion 122,

Expandable nosecone 190 is configured to transition between expanded and collapsed configurations thereof, and is connected to a nosecone shaft 192 defining a guidewire lumen, through which retrieval guidewire 128 can extend. Nosecone shaft 192 extends through a lumen of retrieval shaft 120^{j}. the nosecone shaft 192 and retrieval shaft 120^{j} are axially movable relative to each other. The expandable nosecone 190 can have a barrel-like configuration in its expanded state, defining a distal tapering portion 194 extending from a narrow distal tip to a larger diameter in the proximal direction.

The configuration shown in FIG. 10A can be representative of a delivery configuration of embolus removal apparatus 100ⁱ, during which the distal portion 122ⁱ of retrieval shaft 120^{j} can be aligned with or distal to the distal end 106 of delivery catheter 102, and the expandable nosecone 190 is expanded against the distal portion 122ⁱ, such that the distal tapering portion 194 of the expandable nosecone 190 extends distally from the distal portion 122ⁱ, optionally with the largest diameter of the expanded nosecone 190 pressed against the walls at the distal end of the distal portion 122ⁱ of retrieval shaft 120ⁱ. This configuration allows expanded nosecone 190 to serve as a continuation of distal portion 122ⁱ, so as to facilitate safer and easier advancement of the embolus removal apparatus 100ⁱ through the patient's vasculature.

In some examples, expandable nosecone 190 includes elongated slits 196 defining a plurality of ribs 198 therebetween, along the circumference of the nosecone 190. The ribs 198 may be made of a shape-memory material, shape-set to assume a curved or other appropriate configuration extending radially-outward in a free state thereof. Expandable nosecone 190 can be advanced through a lumen of retrieval shaft 120^{j} in a collapsed or compressed configuration, such as due to the ribs 198 of expandable nosecone 190 being forced to assume an oblong or other collapsed configuration by the bounding walls of the retrieval shaft 120^{j}, particularly, for example, along main retrieval shaft body 126. When the nosecone shaft 192 is axially pushed relative to the retrieval shaft 120^{j}, placing the nosecone 190 at or past the wider distal portion 122^{j}, the nosecone 190 is no longer circumferentially restricted, and is free to assume its expanded configuration, as illustrated for example in FIG. 10A.

It is to be understood that an expandable nosecone 190 can be implemented to be actively expandable and collapsible, not necessarily due to self-expansion of pre-shaped ribs 198 thereof. For example, an expandable nosecone can include an actuation mechanism by which the proximal and distal end portions of the nosecone can be approximated towards each other, thereby forcing ribs or struts of the nosecone to protrude radially outwards, and when the ends of the nosecone are distanced away from each other, the nosecone can assume its collapsed configuration. In some examples, an expandable nosecone can inflatable and deflatable, such that injection of inflation fluid, such as saline or any other appropriate liquid or gas, can be injected into a cavity of the nosecone to cause inflation thereof, in which case the nosecone can be shaped to assume a desired shape defining a distal tapering portion in its expanded and inflated shape, and can be deflated by suction of the inflation fluid, thus transitioning to the collapsed configuration.

As shown in FIG. 10B, the distal portion of embolus removal apparatus 100^{j} can be further advanced towards the embolus 70, for example approximating the tip of the nosecone closer to embolus 70. The collection basket 140^{j} can be deployed out of delivery catheter lumen 104 and assume its expanded configuration, in a manner similar to that described for collection basket 140^{d} with respect to FIGS. 6A-6D. In some examples, collection basket 140^{j} can include a closing mechanism 160, schematically illustrated in FIGS. 10A-10D to include cover flaps 164 defining narrow opening 162 therebetween, similar to exemplary collection basket 140^{d} with respect to FIGS. 6A-6D. It is to be understood, as stated above, that collection basket 140^{j} can include any other type of closing mechanism 160, such as any exemplary closing mechanism 160 described above with respect to FIGS. 7A-8E.

Prior to capturing embolus 70 and coupling it to distal portion 122^{j}, expandable nosecone 190 is collapsed and retracted from distal portion 122^{j}, as shown in FIG. 10C. This can be achieved by proximally pulling the nosecone shaft 192 relative to the retrieval shaft 120^{j}, forcing the expandable nosecone 190 to assume a narrower collapsed profile as it is inserted into the narrower diameter of the main retrieval shaft body 126, when the expandable nosecone is formed to include self-expandable ribs 198 for example, or by actively collapsing the nosecone, such as by distancing the proximal and distal ends of the nosecone from each other or by deflating an inflatable nosecone, while still positioned in or distal to the distal portion 122^{j}, after which the nosecone shaft can be axially pulled to insert the collapsed nosecone back inside the main retrieval shaft body 126. Selectively expanding and collapsing an expandable nosecone can provide nosecone functionality when the nosecone is expanded during delivery of the apparatus 100ⁱ through the patient's vasculature, yet removed from the distal position between the distal portion 122^{j} and a target embolus 70, to exposed an uninterrupted pathway along which the embolus 70 can be captured and coupled to the distal portion 122^{j}.

In some examples, the expandable nosecone 190 defines a barrel-like shape (parabolic ellipsoid). In some examples, the expandable nosecone includes a proximal tapering portion in addition to a distal tapering portion, the proximal tapering portion narrowing to a smaller diameter of the nosecone shaft 192 in the proximal direction. A distal tapering portion 194 can be shaped, in some examples, to define an obtuse angle relative to a longitudinal axis of the nosecone, to case advancement of nosecone 190 through the patient's vasculature. A proximal tapering portion of the expandable nosecone can be shaped, in some examples, to define an obtuse angle relative to a longitudinal axis of the nosecone, to ease to case the withdrawal of nosecone 190 into a narrower diameter of the main retrieval shaft body 126.

In some examples, a distal tip of the nosecone, surrounding the exit opening through which the guidewire 128 can extend, is formed as an atraumatic tip or end portion. In some examples, the outer surface of inflatable nosecone 190 is relatively smooth, to minimize friction while advanced through the patient's vasculature and/or to reduce friction with the inner surface of retrieval shaft 120^{j} when axially translated through the retrieval shaft 120^{j}. Electropolish and/or mechanical polish can be utilized during manufacturing to smooth the surface of inflatable nosecone 190, such as an outer surface of ribs 198. In some examples, inflatable nosecone 190 can be coated by a lubricant or a low-friction layer to minimize friction, such as Teflon, Silicone coating, or any other low friction coating known to those skilled in the art. According to some examples, inflatable nosecone 190 can be coated by a hydrophilic coating or a hydrophobic coating.

The ribs 198 illustrated and described above with respect to inflatable nosecone 190 can be also referred to as struts. Any number of struts or ribs 198 is contemplated. Formation of a nosecone 190 including ribs of struts 198 separated by slits 196 can be achieved by forming such slits or cutouts along a tube from which the nosecone 190 can be formed, such as by laser cutting, chemical etching, water jet cutting, blade or knife cutting, or any other suitable cutting technique. In some examples, expandable nosecone 190 is pre-formed in the expanded configuration during the manufacturing procedure. This can be achieved by cutting struts of ribs 198 from a sheet of material, such as by laser cutting, chemical etching, water jet cutting, blade or knife cutting, or any other suitable cutting technique. The cut ribs can be then rolled into a tubular configuration, interconnected by welding, gluing, or any other affixing or bonding technique known in the art, which is also biocompatible.

In some examples, a nosecone 190 comprising a plurality of ribs 198 separated by slits 196 can be thermally treated to shape-set the ribs 198 to a desired expanded shape, as is known to those skilled in the art of thermally setting shape-memory materials, such as nickel titanium or polymeric materials. Thermal treatment of nosecone 190 may include, in addition to shape setting, a treatment to instill super elastic material behavior of the nickel titanium (or other) alloy within the relevant temperature range. Following such thermal treatment, expandable nosecone 190 may assume a desired expanded shape at body temperature. In some examples, the transition temperature of the nickel titanium (or other suitable) alloy may be set to a temperature below the body temperature, but above storage temperature (e.g. between about 25° C. and about 36° C.). Such programing of the transition temperature can advantageously minimize the force applied by the nosecone 190 while the apparatus is in storage. In some examples, the transition temperature can be set below the storage temperature.

In some examples, the ribs 198 can be made of wires or ribbons which are secured together on at least one side or preferably on both distal and proximal ends of the resulting nosecone 190. Such securement can be achieved by welding, soldering, gluing, crimping, using a shrink, of any other suitable fixation procedure. For such a design, a variety of elastic materials can be used, including, but not limited to: metals (e.g. Elgiloy, stainless steel, steel, copper, titanium, nickel titanium), or polymers (e.g. silicone, polyurethane, PET, Teflon, PEEK, Polyimide, Polyamide, or other suitable biocompatible polymers).

The ribs 198 can have a round or circular cross sectional shape, or any other cross-sectional shape, such as: rectangular, squared, triangular, oval, diamond-shaped, pentagonal, hexagonal, curvilinear, and the like. The cross sectional shape of the struts or ribs 198 may be important for minimization of trauma or injury to the blood vessels or other body structures while advancing nosecone 190 in an expanded configuration through the patient's vasculature.

As mentioned, the distal tapering portion 194 expands from a narrow diameter at the distal tip of the nosecone 190, to a greater diameter at the proximal end of distal tapering portion 194 (which can be an intermediate portion between the proximal and distal ends of nosecone 190). In some examples, the outer diameter of the proximal end of the distal tapering portion 194 is smaller than the inner diameter of the diameter of the distal portion 122^{j} (at its widest portion) by no more than 25%, nor more than 20%, nor more than 15% nor more than 10%, or nor more than 5%. In some examples, the inner diameter of the diameter of the distal portion 122) (at its widest portion) is greater than the outer diameter of the proximal end of the distal tapering portion 194 by at least 5%, by at least 10%, by at least 15%, or by at least 25%.

In some examples, capture mechanism 129 can be implemented as induced suction force, such as illustrated in FIG. 10D, though it is to be understood that any other capture mechanism 129 can be employed, such as by utilizing an embolus-capture element 130 as disclosed herein. When implemented as vacuum suction, sufficient space can be retained between the collapsed expandable nosecone 190 and the surrounding walls of the retrieval shaft 120^{j} to prevent the nosecone 190 from completely blocking passage through the retrieval shaft 120^{j}, and allow vacuum suction to be applied therethrough, for example through spacings that remain along slits 196. In some examples, the expandable nosecone 190 can be retracted to a position far enough from the distal portion 122^{j} or fully removed from retrieval shaft 120^{j} to prevent it from blocking a pathway through which vacuum suction is applied.

As further illustrated in FIG. 10D, the retrieval shaft 120^{j} can be retracted so as to position the captured embolus 70 inside basket cavity 148, and released from the distal portion 1221 after which exemplary steps can be performed according to any manner similarly described for embolus removal apparatus 100^{d} with respect to FIGS. 6A-6D. It is to be understood that while deployment of the basket 140^{j} is illustrated in FIGS. 10A-10D to be performed prior to collapsing and retracting expandable nosecone 190, this is not meant to be limiting, and that any order of some of the steps described above with respect to FIGS. 10A-10D can be altered, such as by collapsing and/or retrieving the expandable nosecone 190 prior to deployment and expansion of basket 140^{j}.

In some examples, the retrieval shaft 120 includes a pre-shaped segment 121 extending along a distal segment thereof, between the distal portion 122 and the main retrieval shaft body 126. FIGS. 11A-11B shows examples of optional pre-shaped segments 121. FIG. 11A shows an exemplary retrieval shaft 120^{k} having a curved pre-shaped segment 121^{k}, such that an C1 axis extending through the distal portion 122^{k} is angled with respect to an axis C2 extending through the main retrieval shaft body 126^{k} at a portion proximate or immediately extending from the pre-shaped segment 121^{k}. FIG. 11B shows an exemplary retrieval shaft 120^{l} having a curved pre-shaped segment 121^{l}, such that the axis C1 extending through the distal portion 122^{l} is perpendicular with respect to the axis C2 extending through the main retrieval shaft body 126^{l} at a portion proximate or immediately extending from the pre-shaped segment 121^{l}. It is to be understood that while two shapes of the pre-shaped segment are illustrated in FIGS. 11A-11B, any other shape is contemplated, configured to result in the axis C1 of the distal portion to be non-parallel with the axis C2 of the main retrieval shaft body 126. Any retrieval shaft 120 of any exemplary embolus removal apparatus 100 disclosed herein can optionally include a pre-shaped segment 121.

The pre-shaped segment 121 can be made of a shape-memory material, such as a shape-memory polymer, pre-shaped to assume a non-straight shape in a free state thereof. The pre-shaped segment 121 can be flexible enough to assume a relatively straight configuration when positioned inside the delivery catheter 102 during advancement through the patient's vasculature, and once deployed or advanced distally from delivery catheter lumen 104, for example, the segment 121 can be free to assume its pre-shaped non-straight configuration. In some examples, a cover-sheath 124, similar for example to cover-sheath 124 shown in FIGS. 9A-9B, can be disposed over segment 121 during delivery of the embolus removal apparatus 100, forcing it to assume a relatively straight configuration, and once deployed or advanced from the cover-sheath 124, the segment 121 can be free to assume its pre-shaped non-straight configuration.

Forming a retrieval shaft 120 to include a pre-shaped segment 121 at a distal portion thereof can advantageously facilitate easier navigation of the retrieval shaft 120 during the procedural steps of capturing and moving an embolus 70. For examples, upon approaching a junction such as the pulmonary trunk 452, illustrated for example in FIG. 1A-1I, retrieval shaft 120 can be advanced distally from delivery catheter 102, oriented such that the distal portion 122 faces toward embolus 70. Once embolus 70 is captured and coupled to the distal portion 122, the retrieval shaft 120 can be rotated, such as around axis C2 of main retrieval shaft body 126, so as to direct the distal portion 122 and the embolus 70 carried thereby, toward the embolus deposit opening 142 of collection basket 140. In some examples, embolus removal apparatus 100 comprises an angular orientation control mechanism, such that the angular orientation of the retrieval shaft 120 can be controlled by the physician, for example by facilitating rotational movement of the retrieval shaft 120 about axis C2 of main retrieval shaft body 126.

In some examples, a retrieval shaft 120 that includes a pre-shaped segment 121 can be navigated between the delivery catheter 102, the embolus 70, and the basket 140, without the use of a retrieval guidewire 128. In some examples, a retrieval shaft 120 that includes a pre-shaped segment 121 can be navigated over a retrieval guidewire 128, in which case the pre-shaped segment 121 can be utilized as a feature that assist in easier and more precise navigation of the retrieval shaft 120.

In some examples, the retrieval shaft 120 further comprises a centering and/or stabilizing mechanism, configured to stabilize it when reaching an embolus 70 to be retrieved. FIG. 10A illustrates an exemplary retrieval shaft 120^{k} that further includes an inflatable stabilizing balloon 125 disposed around a circumference of the retrieval shaft 120^{k} at a position proximal to the distal portion 122^{k}. It is to be understood that though retrieval shaft 120^{k} that includes an inflatable stabilizing balloon 125 is illustrated in FIG. 10A to further include a pre-shaped segment 121, this combination is not mandatory, and that any of the inflatable stabilizing balloon 125 or the pre-shaped segment 121 can be implemented for a retrieval shaft 120 independently. That is to say, a retrieval shaft 120^{k} that includes an inflatable stabilizing balloon 125 can be provided with or without a pre-shaped segment 121. Furthermore, any retrieval shaft 120 of any exemplary embolus removal apparatus 100 disclosed herein can optionally include an inflatable stabilizing balloon 125.

The inflatable stabilizing balloon 125 can define an internal cavity disposed around the outer wall of retrieval shaft 120^{k}, isolated from the lumen of retrieval shaft 120^{k}, such that the lumen of retrieval shaft 120^{k} can define an uninterrupted space through which components, such as retrieval guidewire 128, can extend, and/or through which suction force can be applied. The inflatable stabilizing balloon 125 can be inflated by streaming pressurized inflation fluid, which can be saline or any other suitable liquid or gas, into the internal cavity of the balloon 125, and can be deflated by suction of the inflation fluid from the balloon 125. As further shown in FIG. 11A, an inflation tube 127 can extend into the balloon 125 and have an opening exposed to the internal cavity of the balloon 125. The inflation tube 127 can be connected at a proximal end thereof, external to the patient's body, to an inflation fluid source, such as a syringe and/or a pump. In some examples, the inflation tube 127 can extend through the lumen of retrieval shaft 120^{k} along its length, up to the position of the balloon 125, at which point it can penetrate, through a sealed aperture or passage formed through the wall of retrieval shaft 120^{k}, toward and/or into the internal cavity of inflatable stabilizing balloon 125.

The inflatable stabilizing balloon 125 can be retained in a deflated state during delivery to the site of treatment. Upon approaching a target embolus 70 in a target blood vessel, and optionally prior to utilization of a capture mechanism 129 to capture and couple the embolus 70 to the distal portion 122^{k}, the stabilizing balloon 125 can be inflated by injecting an inflation fluid, through inflation tube 127, into its cavity. The balloon 125 is expanded against the surrounding anatomical walls of the blood vessels, applying sufficient pressure against the surrounding blood vessel to center and stabilize the retrieval shaft 120^{k} in position, preventing undesired accidental movement thereof relative to the blood vessel or the embolus 70. At this stage, any suitable capture mechanism 129 can be employed to capture the embolus and approximate it to contact with the distal lip of the distal portion 122^{k}.

In the absence of a stabilizing element such as balloon 125, the process of capturing and moving of the embolus 70 can apply forces on the retrieval shaft 120^{k} that might displace it, making it harder to adequately couple embolus 70 to distal portion 122^{k}. Thus, inflating a stabilizing balloon 125 can advantageously center and stabilize the retrieval shaft 120^{k}, and in particular, its distal portion 122^{k}, with respect to the blood vessel, thus facilitating the process of capturing and moving the embolus 70 toward the distal portion 122^{k}. Once the embolus 70 is coupled to distal portion 122^{k}, the balloon 125 can be deflated, and the retrieval shaft 120^{k} can be navigated to position the captured embolus in basket 140 according to any of the examples described herein.

FIGS. 12A-13D show exemplary embolus-capture elements 130 that can be utilized with any exemplary embolus removal apparatus 100 disclosed herein. An embolus-capture element 130 can be carried on a distal end portion of a retrieval guidewire 128, as described above with respect to retrieval guidewire 128^{a} for example, or can be carried on distal end portion of a separate independent capture element shaft 131, which can be axially movable through and with respect to retrieval shaft 120, and can be optionally used in combination with a retrieval guidewire 128 that can axially pass through a lumen of a tubular capture element shaft 131, or side by side in parallel to capture element shaft 131, in which case capture element shaft 131 can be implemented as a wire, rod, tube, and the like.

An embolus-capture element 130 can be implemented as an element configured to transition between a collapsed and an expanded configuration. The embolus-capture element 130 can be retained in a collapsed configuration during delivery toward the target embolus 70, and can be passed, in its collapsed configuration, through and past the embolus 70, for example by axially advancing a retrieval guidewire 128 and/or a capture element shaft 131 in which the element 130 is comprised, through the length of the embolus 70. Once positioned distally to the embolus 70, the embolus-capture element 130 can be expanded, such as by actuating a suitable actuation mechanism controllable by the physician.

In some examples, the embolus-capture element 130 is shape-set to passively self-expand to an expanded configuration when not restricted by an external boundary. For example, an embolus-capture element 130 can be retained in a collapsed or partially-expanded configuration when positioned inside the lumen of retrieval shaft 120. The embolus-capture element 130 can be further retained or forced to assume a collapsed configuration during advancement through an embolus 70. The embolus-capture element 130 can have a conical tapering distal portion that gradually constricts it when forced to pass in a distal direction through the matter of the embolus 70. Once passing past embolus 70, the embolus-capture element 130, no longer restricted by an outer shaft and/or tissue material, can be free to self-expand.

In its expanded state, the embolus-capture element 130 can be pulled proximally up to contact with the embolus 70, at which point, further proximal displacement of the embolus-capture element 130 will cause it to dislodge the embolus 70 from the blood vessel and proximally pull it therewith, dur to the increase surface are of the expanded element 130 acting against the embolus 70.

The embolus-capture element 130 is thus utilized to dislocate the embolus 70 and bring it into contact with the distal portion 122 of retrieval shaft 120, by proximally pulling the embolus-capture element 130 relative to retrieval shaft 120, and/or distally pushing the retrieval shaft 120 relative to the embolus-capture element 130, until the embolus is sandwiched between the embolus-capture element 130 and the distal portion 122, thereby coupling the embolus 70 to the distal portion 122. The embolus-capture element 130 can continue application of a proximally-oriented force acting against the embolus 70 during navigation of the retrieval shaft 120 toward and into the basket 140, to prevent accidental release of the embolus 70 during this movement, Once embolus 70 is positioned inside the basket cavity 148, the embolus 70 can be released by distally pushing the embolus-capture element 130 relative to retrieval shaft 120, and/or proximally pulling the retrieval shaft 120 relative to the embolus-capture element 130.

In some examples, embolus-capture element 130 defines, in its expanded state, a maximal outer diameter in the range of about 2 mm to about 30 mm. In some examples, embolus-capture element 130 defines, in its expanded state, length in the range of about 2 mm to about 50 mm.

FIGS. 12A-12B show an exemplary embolus-capture element 130^{m} that includes an expandable plate 132. The expandable plate 132 can be configured to assume a relatively conical or otherwise narrow-shaped collapsed configuration when bound by an outer restricting boundary, such as when disposed inside an overshaft 133 as shown in FIG. 12A, and can expand to a planar or a wider conical or cup-shaped configuration in its free state, as shown in FIG. 12B. As illustrated in FIG. 12B, the expanded area of the plate 132 can be large enough to press against the distal end of an embolus 70 and coupled it to a distal portion 122 of retrieval shaft 120. It is to be understood that a distal portion 122 having a diameter similar to that of the remainder of retrieval shaft 120 is shown throughout FIGS. 12A-13D by way of illustration and not limitation, and that any other exemplary distal portion 122, including tapering or expandable distal portions, are contemplated for used with any exemplary embolus-capture element 130.

The overshaft 133 can be dimensioned to have an inner lumen which is significantly less than the inner diameter of retrieval shaft 120 and/or its distal portion 122, configured to retain the plate 132 in a collapsed or folded configuration prior to being passed through embolus 70. The overshaft 133 can extend over capture element shaft 131 and/or retrieval guidewire 128. A narrow profile of the plate 132 is desirable at the entry into, and during passage through, the embolus 70, to prevent embolus fragmentation as it passed through it.

In some examples, an embolus-capture element 130 can include a plurality of arms, which can be in the form of struts, plates, and the like, attached at their distal ends to the retrieval guidewire 128 and/or a capture element shaft 131, and configured to transition from a collapsed configuration, having their free ends pressed closer to the retrieval guidewire 128 and/or a capture element shaft 131, for example when kept inside an overshaft 133, to an expanded configuration, wherein the arms are free to extend radially away from the a retrieval guidewire 128 and/or a capture element shaft 131. The schematic representation shown in FIGS. 12A-12B can similarly represent, instead of a cross-sectional view of an expandable plate, a view of two of a plurality of arms.

FIG. 13A shows an exemplary embolus-capture element 130^{o} that includes a helical coil 137. This type of embolus-capture element 130^{o} is not necessarily configured to move between expanded and collapsed configuration, but may rather assume a single configuration, such that helical coil 137 can be retained in a retracted position during delivery, within the lumen of retrieval shaft 120, and can be axially advanced in a rotational motion to screw through the embolus 70. When positioned distal to the embolus 70, as shown in FIG. 13A, axially pulling the coil 137 without rotating it, can serve to dislodge and move the embolus 70. The diameter of the coils can be large enough to exert sufficient force to move the embolus 70 and retained it in position against the distal portion 122. When the embolus 70 is positioned in basket cavity 148, the embolus 70 can be released by pulling the helical coil 137 while rotating it in an opposite direction.

FIG. 13B shows an exemplary embolus-capture element 130^{p} that includes an expandable frame 134. The expandable frame 134 include a plurality of struts, which can be a series of longitudinal struts as illustrated, or can be similar to any other type of an expandable stent, for example, configured to self-expand once no longer bound, at a position distal to the embolus 70.

FIG. 13C shows an exemplary embolus-capture element 130^{q} that includes a proximally forked arrangement 135, and can be formed of a plurality of curved struts which are attached at their distal ends to the retrieval guidewire 128 and/or a capture element shaft 131, and extend, in the expanded configuration, proximally to form an umbrella-like profile, such that their free ends are facing the distal end of embolus 70. When pulled in a proximal direction, their free ends can partially penetrate into the embolus 70 to improve engagement therewith. The struts forming the forked arrangement 135 are resilient enough to dislodge and move the embolus 70 towards distal portion 122 when proximally pulled thereagainst.

In some examples, an embolus-capture element 130 can include a proximally forked arrangement as illustrated in FIG. 13C, as well as a distally forked arrangement, oriented opposite to the proximally forked arrangement. A distally forked arrangement can be axially spaced from the proximally forked arrangement, arranged proximally thereto. The distally forked arrangement can have a plurality of curved struts which are attached at their proximal ends to the retrieval guidewire 128 and/or a capture element shaft 131, and extend, in the expanded configuration, distally to form an pitchfork-like profile, such that their free ends are facing the proximal end of embolus 70. When expanded, the two arrangements can be disposed at both axial sides of the embolus, to retain it from both ends.

FIG. 13D shows an exemplary embolus-capture element 130^{r} that includes an inflatable pulling balloon 136. The inflatable pulling balloon 136 can be inflatable and depletable in a similar manner as that of any other inflatable balloon disclosed herein, such as balloon 125 or an inflatable expandable nosecone. While not explicitly illustrated, an inflation tube, similar to inflation tube 127 described above, can extend through or along any of the retrieval guidewire 128 and/or a capture clement shaft 131, utilized so as to inflate and deflate the balloon 136. The inflatable pulling balloon 136 can be passed, while deflated, through the embolus 70, and once position distal to embolus 70, the balloon 136 can be inflated, as illustrated, to a diameter sufficient to dislodge and pull the embolus 70 responsive to pulling of the inflated balloon 136. When the embolus 70 is positioned in basket cavity 148, the embolus 70 can be released by deflating the balloon 136 and pulling it away from the embolus 70.

While not explicitly illustrated in any of FIGS. 13B-13D, it is to be understood that any of the embolus-capture elements 130^{p} or 130^{q}, and in some cases, 130^{r}, can extend through an overshaft 133 of the type illustrated in FIGS. 12A-12B and described above. For example, any of the expandable frame 134 or forked arrangement 135 can be retained in a compacted or collapsed configuration thereof when positioned inside an overshaft 133, in the same manner described above with respect to FIGS. 12A-12B.

FIG. 14 is a flowchart depicting examples a method 600 of removing at least one embolus from at least one blood vessel. As shown in FIG. 14, the method 600 comprises advancing, at 602, a delivery catheter 102 of an embolus removal apparatus 100 through the vasculature of a patient, towards a site of treatment which includes at least one embolus. Any exemplary embolus removal apparatus 100 disclosed above may be utilized in conjunction with the method 600. Advancing the embolus removal apparatus 100 can be performed through any appropriate approach and via any path of the vasculature appropriate for advancement toward a target site of treatment, including advancement paths toward the pulmonary arteries, advancement through the jugular vein towards limp deep veins, or any other examples described above, such as with respect to FIGS. 5A-5B.

The method further comprises extending, at 604, an embolus collection basket 140 past a distal end 106 of the delivery catheter 102 and expanding, at 606, the embolus collection basket 140 distally to the delivery catheter 102. In some examples, the basket 140 can be a self-expandable basket, such that expanding the basket, at 606, is performed simultaneously as the basket is extended at 604. In some examples, the basket 140 can be actively controllable to transition between collapsed and expanded configurations thereof, such that expanding the basket at 606 can be performed subsequent to extending the basket at 604, for example by pushing wire portions 151 similar to those illustrated 3A-4B, having sufficient rigidity to push against the basket and enlarger the diameter of rim 144.

The method further comprises extending, at 608, a retrieval shaft 120 past the distal end 106 of the delivery catheter 102 and towards the at least one embolus 70, and capturing, at 610, the at least one embolus 70 by a capture mechanism 129. The capture mechanism 129 can be implemented according to any example disclosed herein, such as by applying suction force through the retrieval shaft 120 and/or passing an embolus-capture element 130, implemented according to any of the examples described above, through the embolus 70, and utilizing the embolus-capture element 130 to dislodge the embolus 70 and move it toward a distal portion 122 of the retrieval shaft 120.

The method further comprises guiding, at 612, the retrieval shaft 120, with the at least one embolus 70 coupled thereto, towards and into a collection basket cavity 148 of the collection basket 140 through an embolus deposit opening 142. As mentioned, the capture mechanism 129 is utilized to keep the embolus 70 coupled to the distal portion 122 of the retrieval shaft 120 during this movement. The method further comprises retrieving, at 618, the embolus removal apparatus 100 from the patient's body.

In some examples, the method 600 can further comprise, subsequent to the guiding at 612 and prior to retrieving apparatus 100 at 618, releasing, at 614, the at least one embolus 70 from the retrieval shaft 120 inside the collection basket cavity 148. In some examples, the at least one embolus 70 can remain coupled to the retrieval shaft during retrieving apparatus 100 at 618, in which case the releasing at 614, at least with respect to a final embolus 70 captured in the basket 140, is not performed.

In some examples, the method 600 can further comprise, subsequent to the releasing at 614 and prior to retrieving the apparatus 100 at 618, retracting, at 616, the retrieval shaft 120 from the collection basket cavity 148.

In some examples, such as after placing the final embolus 70 inside the basket 140, the method 600 further comprises, prior to retrieving the apparatus 100 at 618, retracting, at 638, the collection basket 140 towards the distal end 106 of the delivery catheter 102. This can be performed such that at least a portion of the collection basket 140 is retained within a lumen 106 of the delivery catheter 102, as illustrated for example in any of FIG. 11 or 6D. Basket retraction can also squeeze emboli 70 retained therein, as described in greater detail above. In some examples, retracting the collection basket 140 at 638 can be performed subsequent to any of: guiding the retrieval shaft 120 to the basket 140 at 612, releasing the embolus 70 at 614, or retracting the retrieval shaft 120 at 618.

In some examples, the method 600 further comprises, prior to retracting the apparatus 100 at 618, checking, at 640, whether another embolus 70 needs to be retrieved. The checking at 640 can be performed subsequent to retraction of the retrieval shaft 120 from the basket 140 at 616. If no additional embolus 70 needs to be retrieved, the method 600 can proceed to retrieval of the apparatus 100 at 618. If another embolus 70 needs to be retrieved, the method can repeat extending the retrieval shaft, at 608, to ward the additional embolus 70, and any of the subsequent steps described herein, such as capturing the additional embolus 70 at 610, guiding at 612, optionally releasing the additional embolus at 614, and optionally retracting the retrieval shaft at 616.

In some examples, expanding the embolus collection basket 140 at 606 comprises expanding the collection basket 140, at 630, such that an embolus deposit opening 142 is at the proximal end of basket 140, as illustrated, for example, in FIGS. 1A-3C. In such cases, expansion of the basket 140 at 630 can be in a branch or blood vessel which is different from that in which the target embolus 70 is disposed. For example, in such cases, extending the embolus collection basket 140 past the distal end 106 of delivery catheter 102 at 604 comprises positioning the embolus collection basket 140, at 605, in a contralateral blood vessel relative to that of the embolus 70. Furthermore, the retrieval shaft 120 can be extended and directed in such cases, at 608, in a direction different than the direction in which the basket 140 was extended at 604, including the contralateral direction.

In some examples, expanding the embolus collection basket 140 at 606 comprises expanding the collection basket 140, at 632, such that an embolus deposit opening 142 is at the distal end of basket 140, as illustrated, for examples, in FIGS. 6A-6D or 10A-10D. In such cases, the retrieval shaft 120 can extend, at 608, through the basket 140.

In some examples, when the embolus removal apparatus 100 includes an expandable nosecone 190, the method 600 further comprises, subsequent to extending the retrieval shaft 120 at 608 and prior to capturing the embolus 70 at 610, collapsing, at 634, the expandable nosecone, and retrieving it proximally into the lumen of retrieval shaft 120, as described with respect to FIGS. 10A-10D for example. In such cases, advancing the embolus removal apparatus 100 at 602 can be performed while the expandable nosecone 190 is in the expanded configuration, with a distal tapering portion 194 thereof extending distally from the distal portion 122 of retrieval shaft 120.

In some examples, when the embolus collection basket 140 further comprises a closing mechanism 160, releasing the embolus 70 in basket cavity 148 at 614 further comprises closing, at 636, at least a portion of the embolus deposit opening 142 using the closing mechanism 160, wherein the closing mechanism can be implemented according to any of the examples described above with respect to FIGS. 7A-8E, for example.

In some examples, the method 600 can further include subsequent to guiding retrieval shaft 120 with embolus 70 into the basket cavity 148, confirming that the embolus 70 is captured in the basket cavity 148. This can be performed at any stage prior to retrieving the embolus removal apparatus 100 at 618, for example by injecting suitable contrast into the basket cavity 148 and imaging, such as via fluoroscopy or other appropriate imaging modality, the region of the basket to verify that the embolus 70 is properly retained therein.

In some examples, capturing the embolus 70 at 610 further comprises verifying that the embolus 70 is coupled to the distal portion 122 of retrieval shaft 120. For example, when the capture mechanism 129 is implemented as application of vacuum or suction force, such as by pulling a plunger of a syringe connected to retrieval shaft 120, a pulling force will act on the plunger of the syringe during suction if the embolus 70 seals the outer of retrieval shaft 120. This force can be identified and/or measured to verify that the vacuum indeed pulls and couples the embolus 70 to the distal portion 122 of retrieval shaft 120.

### SOME EXAMPLES OF THE DISCLOSED IMPLEMENTATIONS

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. An embolus removal apparatus comprising:
a delivery catheter defining a delivery catheter lumen;
an embolus collection basket, coupled to a basket shaft which extends through the delivery catheter lumen, the embolus collection basket comprising porous walls that surround a collection basket cavity, extending between an embolus deposit opening and a permanently closed end;
a retrieval shaft extending through the delivery catheter lumen, comprising a distal portion; and
at least one capture mechanism configured to couple an embolus to the distal portion of the retrieval shaft;
wherein the retrieval shaft is axially movable relative to the delivery catheter,
wherein the basket shaft is axially movable relative to the delivery catheter,
wherein the collection basket is configured to transition between a collapsed state and an expanded state;
wherein the retrieval shaft is movable between a first position of its distal portion distanced from the collection basket cavity to a second position of its distal portion inside the collection basket cavity; and
wherein the at least one capture mechanism is configured to maintain engagement between an embolus and the distal portion of the retrieval shaft during translation of the distal portion from the first position to the second position.

Example 2. The apparatus of any example herein, particularly example 1, wherein the at least one capture mechanism comprises suction force applied through the distal portion of the retrieval shaft.

Example 3. The apparatus of any example herein, particularly example 1, wherein the distal portion of the retrieval shaft comprises an active electrode and a reference electrode, and wherein the at least one capture mechanism comprises positive voltage applied between the active electrode and the reference electrode.

Example 4. The apparatus of any example herein, particularly example 1, wherein the at least one capture mechanism comprises cryogenic cooling applied at the distal portion of the distal portion of the retrieval shaft.

Example 5. The apparatus of any example herein, particularly any one of examples 1 to 4, wherein the collection basket is self-expandable, configured to transition to its expanded configuration upon being deployed out of the delivery catheter lumen.

Example 6. The apparatus of any example herein, particularly any one of examples 1 to 5, wherein the distal portion of the retrieval shaft is flared radially outward in the distal direction.

Example 7. The apparatus of any example herein, particularly any one of examples 1 to 5, wherein the distal portion of the retrieval shaft is configured to transition between collapsed and expanded configurations thereof.

Example 8. The apparatus of any example herein, particularly any one of examples 1 to 7, wherein the retrieval shaft further comprises an inflatable stabilizing balloon around a portion of the retrieval shaft, proximal to the distal portion of the retrieval shaft.

Example 9. The apparatus of any example herein, particularly any one of examples 1 to 8, wherein the embolus deposit opening is at a proximal end of the embolus collection basket.

Example 10. The apparatus of any example herein, particularly example 9, wherein the retrieval shaft does not extend through the basket shaft.

Example 11. The apparatus of any example herein, particularly example 9 or 10, wherein the retrieval shaft and the basket shaft are configured to extend toward opposite directions, out of the delivery catheter.

It is to be understood that any reference to the retrieval shaft and the basket shaft being configured to extend toward opposite directions out of the delivery catheter, is not meant to restrict their movement such that the retrieval shaft and the basket shaft must extend in opposite directions, but merely that if and when required, it is possible to extend each in a direction that can be different and even opposite to the direction of the other. That is to say, this does not preclude the ability of the retrieval shaft and the basket shaft to extend out of the delivery catheter in any other direction, including parallel to each other.a

Example 12. The apparatus of any example herein, particularly any one of examples 9 to 11, wherein the basket shaft is connected to a rim disposed around the embolus deposit opening.

Example 13. The apparatus of any example herein, particularly example 12, wherein the rim is angled at an obtuse angle relative to the basket shaft.

Example 14. The apparatus of any example herein, particularly example 12 or 13, wherein the rim is self-expandable.

Example 15. The apparatus of any example herein, particularly any one of examples 9 to 14, wherein the embolus deposit opening is configured to transition between an open state and a closed state.

Example 16. The apparatus of any example herein, particularly any one of examples 12 to 15, wherein the basket shaft comprises at least one wire portion extending toward the rim.

Example 17. The apparatus of any example herein, particularly example 16, wherein the rim and the at least one wire portions are integrally formed as a continuous single wire.

Example 18. The apparatus of any example herein, particularly example 16 or 17, wherein the at least one wire portion comprises two wire portions parallel to each other.

Example 19. The apparatus of any example herein, particularly any one of examples 16 to 18, wherein the basket shaft further comprises a collection basket cover-sheath disposed around the at least one wire portion.

Example 20. The apparatus of any example herein, particularly any one of examples 9 to 19, wherein the retrieval shaft further comprises a pre-shaped segment, configured to transition between a straight configuration and a non-straight configuration.

Example 21. The apparatus of any example herein, particularly any one of examples 9 to 20, further comprising a tether extending through an eyelet of the embolus collection basket, the tether attached at a distal end thereof to the distal portion of the retrieval shaft.

Example 22. The apparatus of any example herein, particularly example 21, wherein the tether is axially movable through the eyelet, and is configured to pull the distal portion of the retrieval shaft towards the collection basket cavity when the tether is proximally pulled.

Example 23. The apparatus of any example herein, particularly any one of examples 1 to 8, wherein the embolus deposit opening is at a distal end of the embolus collection basket.

Example 24. The apparatus of any example herein, particularly example 23, wherein the retrieval shaft extends through a lumen of the basket shaft.

Example 25. The apparatus of any example herein, particularly example 23 or 24, further comprising an expandable nosecone, configured to transition between collapsed and expanded configurations thereof.

Example 26. The apparatus of any example herein, particularly example 25, wherein the expandable nosecone is axially movable with respect to the retrieval shaft.

Example 27. The apparatus of any example herein, particularly example 25 or 26, wherein the expandable nosecone is configured to be positioned, in its collapsed configuration, within a lumen of the retrieval shaft.

Example 28. The apparatus of any example herein, particularly any one of examples 23 to 27, wherein the basket shaft is attached to an end of the embolus collection basket opposite to the embolus deposit opening.

Example 29. The apparatus of any example herein, particularly any one of examples 23 to 28, wherein the embolus collection basket further comprises a basket cover disposed around the basket walls, the basket cover extending distally along a length Lc from the permanently closed end, wherein Lc is less that a length of the basket Lb.

Example 30. The apparatus of any example herein, particularly example 29, wherein Lc is equal to or less than 50% of Lb.

Example 31. The apparatus of any example herein, particularly any one of examples 1 to 30, wherein the embolus collection basket comprises a porous wall extending distally from the basket shaft to a widest end portion of the embolus collection basket, and a an inverted neck extending proximally from the widest end portion to the embolus deposit opening.

Example 32. The apparatus of any example herein, particularly example 31, wherein the diameter of the embolus deposit opening is less than the diameter of the widest end portion.

Example 33. The apparatus of any example herein, particularly example 31 or 32, wherein the inverted neck comprises a stretchable material.

Example 34. The apparatus of any example herein, particularly any one of examples 1 to 33, wherein the embolus collection basket further comprises a closing mechanism, configured to transition the embolus deposit opening between an open state and a closed state.

Example 35. The apparatus of any example herein, particularly example 34, wherein the closing mechanism comprises a narrow opening, sized to allow passage of at least a portion of the retrieval shaft therethrough in the closed state of the embolus deposit opening.

Example 36. The apparatus of any example herein, particularly example 34 or 35, wherein the closing mechanism comprises a plurality of cover flaps overlaying the embolus deposit opening.

Example 37. The apparatus of any example herein, particularly example 36, wherein the cover flaps taper in a direction away from the embolus deposit opening and radially inward toward each other.

Example 38. The apparatus of any example herein, particularly example 36 or 37, wherein the cover flaps are connected to the embolus collection basket by springs configured to bias the cover flaps to the closed state of the embolus deposit opening.

Example 39. The apparatus of any example herein, particularly example 34 or 35, wherein the closing mechanism comprises a drawstring connected to the embolus collection basket around a circumference of the embolus deposit opening, and extending axially therefrom along a length of the embolus collection basket, wherein the drawstring is configured to cinch the embolus collection basket around the embolus deposit opening when proximally pulled.

Example 40. The apparatus of any example herein, particularly example 39, further comprising a drawstring cover shaft disposed over at least a portion of the drawstring.

Example 41. The apparatus of any example herein, particularly example 34 or 35, wherein the closing mechanism comprises a hinged lid, pivotable about a hinge by which it is connected to the embolus collection basket.

Example 42. The apparatus of any example herein, particularly example 41, wherein the closing mechanism further comprises a lid spring configured to bias the hinged lid to the closed state of the embolus deposit opening.

Example 43. The apparatus of any example herein, particularly example 41 or 42, wherein the closing mechanism further comprises an actuation wire attached to the hinged lid, and configured to cause pivotable movement of the hinged lid.

Example 44. The apparatus of any example herein, particularly any one of examples 1 to 43, wherein the at least one capture mechanism comprises an embolus-capture element.

Example 45. The apparatus of any example herein, particularly example 44, wherein the embolus-capture element is comprised in a distal portion of a retrieval guidewire extendable through the retrieval shaft.

Example 46. The apparatus of any example herein, particularly example 44, wherein the embolus-capture element is comprised in a distal portion of a capture element shaft extendable through the retrieval shaft.

Example 47. The apparatus of any example herein, particularly any one of examples 44 to 46, wherein the embolus-capture element is configured to pass through an embolus, and when proximally pulled while positioned within or distal to the embolus, the embolus-capture element is configured to exert a proximally directed pull force against the embolus.

Example 48. The apparatus of any example herein, particularly any one of examples 44 to 47, wherein the embolus-capture element comprises a helical coil.

Example 49. The apparatus of any example herein, particularly any one of examples 44 to 47, wherein the embolus-capture element is configured to transition between a collapsed state and an expanded state.

Example 50. The apparatus of any example herein, particularly example 49, wherein the embolus-capture element comprises an expandable plate.

Example 51. The apparatus of any example herein, particularly example 49, wherein the embolus-capture element comprises an expandable frame.

Example 52. The apparatus of any example herein, particularly example 49, wherein the embolus-capture element comprises a proximally forked arrangement.

Example 53. The apparatus of any example herein, particularly example 49, wherein the embolus-capture element comprises an inflatable pulling balloon.

Example 54. The apparatus of any example herein, particularly any one of examples 49 to 53, further comprising an overshaft extending through the retrieval shaft, wherein the embolus-capture element is axially movable through the overshaft, and wherein the overshaft is configured to retain the embolus-capture element in its collapsed state when the embolus-capture element is positioned inside the overshaft.

Example 55. A method for removing at least one embolus from at least one blood vessel, the method comprising:
advancing a delivery catheter of an embolus removal apparatus towards a site of treatment which includes at least one embolus;
extending an embolus collection basket past a distal end of the delivery catheter; expanding the embolus collection basket distally to the delivery catheter;
extending a retrieval shaft past the distal end of the delivery catheter and towards the at least one embolus;
capturing the at least one embolus by a capture mechanism; guiding the retrieval shaft with the at least one embolus towards and into a collection basket cavity of the collection basket through an embolus deposit opening; and
retrieving the embolus removal apparatus.

Example 56. The method of any example herein, particularly example 55, wherein the expanding the embolus collection basket is automatically performed simultaneously with the extending the embolus collection basket.

Example 57. The method of any example herein, particularly example 55 or 56, wherein the embolus collection basket is self-expandable.

Example 58. The method of any example herein, particularly example 55, wherein the expanding the embolus collection basket is actively performed by an operator of the embolus removal apparatus.

Example 59. The method of any example herein, particularly any one of examples 55 to 58, wherein the capturing the at least one embolus comprises utilizing at least one capture mechanism to couple the at least one embolus to the retrieval shaft.

Example 60. The method of any example herein, particularly example 59, wherein the utilizing at least one capture mechanism comprises applying suction force through the retrieval shaft.

Example 61. The method of any example herein, particularly example 59, wherein the utilizing at least one capture mechanism comprises applying voltage between two electrodes of the retrieval shaft.

Example 62. The method of any example herein, particularly example 59, wherein the utilizing at least one capture mechanism comprises cooling a portion of the retrieval shaft, in close proximity to the embolus, to a cryogenic temperature.

Example 63. The method of any example herein, particularly any one of examples 59 to 62, wherein the utilizing at least one capture mechanism comprises extending an embolus-capture element through the at least one embolus, and proximally pulling the embolus-capture element, thereby pressing the at least one embolus towards and against the retrieval shaft.

Example 64. The method of any example herein, particularly any one of examples 55 to 63, further comprising, subsequent to the guiding the retrieval shaft, releasing the at least one embolus inside the collection basket cavity.

Example 65. The method of any example herein, particularly any one of examples 64, further comprising, subsequent to the releasing the at least one embolus, retracting the retrieval shaft from the collection basket cavity.

Example 66. The method of any example herein, particularly any one of examples 55 to 65, further comprising, prior to the retrieving the embolus removal apparatus, checking whether at least one additional embolus needs to be removed, and if so:
extending the retrieval towards the at least one additional embolus;
capturing the at least one additional embolus by the capture mechanism; and
guiding the retrieval shaft with the at least one additional embolus towards and into the collection basket cavity through the embolus deposit opening.

Example 67. The method of any example herein, particularly any one of examples 55 to 66, further comprising, prior to the retrieving the embolus removal apparatus, retracting the collection basket into the delivery catheter, such that at least a portion of the collection basket extends into a lumen of the delivery catheter.

Example 68. The method of any example herein, particularly example 67, wherein the retracting the collection basket comprises squeezing the at least one embolus.

Example 69. The method of any example herein, particularly any one of examples 55 to 68, wherein the embolus deposit opening is at a proximal end of the collection basket.

Example 70. The method of any example herein, particularly example 69, wherein the extending the retrieval shaft comprises directing the retrieval shaft toward a blood vessel which is different from the blood vessel in which the collection basket is expanded.

Example 71. The method of any example herein, particularly example 69 or 70, wherein the extending the retrieval shaft comprises directing the retrieval shaft toward a contralateral blood vessel, with respect to a blood vessel in which the collection basket is expanded.

Example 72. The method of any example herein, particularly any one of examples 69 to 71, wherein the extending the retrieval shaft is performed such that the retrieval shaft does not pass through the collection basket.

Example 73. The method of any example herein, particularly any one of examples 69 to 72, wherein the guiding the retrieval shaft comprises proximally pulling a tether attached to the retrieval shaft and passing through an eyelet of the collection basket.

Example 74. The method of any example herein, particularly any one of examples 69 to 73, wherein the retrieval shaft comprises a pre-shaped segment, and wherein the guiding the retrieval shaft comprises rotating the retrieval shaft.

Example 75. The method of any example herein, particularly any one of examples 55 to 68, wherein the embolus deposit opening is at a distal end of the collection basket.

Example 76. The method of any example herein, particularly example 75, wherein the extending the retrieval shaft comprises advancing the retrieval shaft through the collection basket.

Example 77. The method of any example herein, particularly example 75 or 76, further comprising, prior to the advancing a delivery catheter, expanding an expandable nosecone such that a distal tapering portion of the nosecone extends distally to the retrieval shaft.

Example 78. The method of any example herein, particularly example 77, further comprising, subsequent to the extending a retrieval shaft and prior to the capturing the at least one embolus, collapsing the expandable nosecone.

Example 79. The method of any example herein, particularly example 78, wherein the collapsing the expandable nosecone comprises retracting the expandable nosecone into a lumen of the retrieval shaft.

Example 80. The method of any example herein, particularly any one of examples 55 to 79, wherein the guiding the retrieval shaft with the at least one embolus into the collection basket cavity comprises transitioning the embolus deposit opening to an open state thereof, through which the at least one embolus can pass into the collection basket cavity.

Example 81. The method of any example herein, particularly example 80, further comprising, subsequent to the guiding the retrieval shaft with the at least one embolus into the collection basket cavity, transitioning the embolus deposit opening to a closed state thereof, through which escape of the at least one embolus from the collection basket cavity is prevented.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1.-81. (canceled)

82. An embolus removal apparatus comprising:
a delivery catheter defining a delivery catheter lumen;
an embolus collection basket, coupled to a basket shaft which extends through the delivery catheter lumen, the embolus collection basket comprising porous walls that define a collection basket cavity, extending between an embolus deposit opening and a permanently closed end;
a retrieval shaft axially extendable through the embolus collection basket, the retrieval shaft comprising a main retrieval shaft body and distal portion; and
wherein the retrieval shaft is axially movable relative to the basket shaft and is not affixed to the embolus collection basket,
wherein the basket shaft is axially movable relative to the delivery catheter,
wherein the collection basket is configured to transition between a collapsed state and an expanded state;
wherein the retrieval shaft is movable between a first position of its distal portion distal to the collection basket cavity to a second position of its distal portion inside the collection basket cavity; and
wherein the retrieval shaft is configured to apply suction force through its distal portion, so as to maintain engagement between an embolus and the distal portion of the retrieval shaft during axial translation of the distal portion.

83. The apparatus of claim 82, wherein the collection basket is self-expandable, configured to transition to its expanded configuration upon being deployed out of the delivery catheter lumen.

84. The apparatus of claim 82, wherein the distal portion of the retrieval shaft defines an inner diameter that is greater than an inner diameter of the main retrieval shaft body.

85. The apparatus of claim 82, wherein the embolus deposit opening is at a distal end of the embolus collection basket.

86. The apparatus of claim 82, wherein the basket shaft is attached to an end of the embolus collection basket opposite to the embolus deposit opening.

87. The apparatus of claim 82, wherein the embolus collection basket further comprises a closing mechanism, configured to transition the embolus deposit opening between an open state and a closed state in the expanded state of the embolus collection basket.

88. The apparatus of claim 87, wherein the closing mechanism comprises a drawstring connected to the embolus collection basket around a circumference of the embolus deposit opening, and extending axially therefrom along a length of the embolus collection basket, and wherein the drawstring is configured to cinch the embolus collection basket around the embolus deposit opening when proximally pulled.

89. The apparatus of claim 88, further comprising a drawstring cover shaft disposed over at least a portion of the drawstring.

90. The apparatus of claim 82, further comprising an embolus-capture element at a distal end of a capture element shaft which is axially movable relative to the retrieval shaft.

91. The apparatus of claim 90, wherein the capture element shaft is configured to pass through an embolus.

92. The apparatus of claim 90, wherein the embolus-capture element is configured to be positioned at least partially distal to the embolus, and when proximally pulled while positioned distal to the embolus, the embolus-capture element is configured to exert a proximally directed pull force against the embolus.

93. The apparatus of claim 90, wherein the embolus-capture element is configured to transition between a collapsed state and an expanded state.

94. The apparatus of claim 93, wherein the embolus-capture element comprises an expandable frame.

95. The apparatus of claim 93, further comprising an overshaft axially movable relative to the embolus-capture element, wherein the overshaft is configured to retain the embolus-capture element in its collapsed state when the embolus-capture element is positioned inside the overshaft.

96. A method for removing at least one embolus from at least one blood vessel, the method comprising:
advancing a delivery catheter of an embolus removal apparatus towards a site of treatment which includes an embolus;
exposing an embolus collection basket out of the delivery catheter;
expanding the embolus collection basket distally to the delivery catheter;
extending a retrieval shaft through the embolus collection basket and towards the embolus;
capturing the embolus by applying suction force through the retrieval shaft configured to maintain engagement between the embolus and the distal portion of the retrieval shaft;
guiding the retrieval shaft with the embolus towards and into a collection basket cavity of the collection basket through an embolus deposit opening;
cinching the embolus deposit opening to a size smaller than that of the embolus; and
retrieving the embolus removal apparatus.

97. The method of claim 96, further comprising, prior to the capturing the embolus, positioning an embolus-capture element at least partially distal to the embolus.

98. The method of claim 97, wherein the capturing the embolus further comprises expanding the embolus-capture element at a position at least partially distal to the embolus, and proximally pulling the embolus-capture element.

99. The method of claim 96, wherein the retracting the collection basket comprises removing the embolus collection basket with the embolus from a patient's body via an introducer sheath.

100. The method of claim 96, wherein the retracting the collection basket comprises squeezing the embolus.

101. The method of claim 96, wherein the cinching comprises pulling a drawstring.
